(19) United States
(12) Patent Application Publication
(10) Pub. No.: US 2021/0290973 A1
(43) Pub. Date: Sep. 23, 2021

(54) CIRCADIAN OPTIMIZED POLYCHROMATIC LIGHT

(71) Applicant: CIRCADIAN ZIRCLIGHT INC., Stoneham, MA (US)

(72) Inventors: Martin Moore-Ede, Wellesley, MA (US); Doros Platika, Charleston, MA (US); Anneke Heitmann, Arlington, MA (US); Rainer Guttkuhn, Maynard, MA (US); John Luciani, Wakefield, MA (US)

(21) Appl. No.: 17/260,215

(22) PCT Filed: Jul. 12, 2019
(86) PCT No.: PCT/US19/41728

§ 371 Date: Jan. 13, 2021

Related U.S. Application Data
(60) Provisional application No. 62/697,911, filed on Jul. 13, 2018.

(51) Int. Cl. A61N 5/06; A61M 21/02

(52) U.S. Cl. CPC A61N 5/0618; A61M 2021/0044; A61M 21/02

(57) ABSTRACT

Methods and apparatus for circadian lighting are disclosed. In particular, improved lighting is described in relation to a novel definition of an optimum relative circadian spectral sensitivity distribution (“Circadian Potency SSD”) with a peak sensitivity at approximately 470 nm-480 nm (preferable embodiments at 472 nm or 478 nm) with a full width at half maximum (“FWHM”) of approximately 50 nm which is skewed asymmetrically between approximately 440 nm and approximately 490 nm. Methods and approaches to determine the Circadian Potency of light sources from their spectral power distributions (SPD) and use the CircadianPotency SSD to optimize healthy circadian function under normal indoor and outdoor illumination conditions are described herein, and incorporated into various light sources, including white light sources, dimmable light sources, and daytime/nighttime light sources.

### CROSS-REFERENCE TO RELATED APPLICATIONS

This application claims the benefit of priority to U.S. Provisional Patent Application Ser. No. 62/697,911, filed Jul. 13, 2018, entitled “CIRCADIAN OPTIMIZED POLYCHROMATIC WHITE LIGHT”, hereby incorporated by reference in its entirety.

### STATEMENT OF GOVERNMENT SUPPORT

This invention was made with Government support under grant number HL110769 awarded by the National Institutes of Health. The Government has certain rights in the invention.

### FIELD

The present disclosure generally relates to the field of illumination, and more particularly, apparatus and methods for providing an improved light in relation to human circadian function.

### INTRODUCTION

The human body is regulated by a circadian timing system which is biologically optimized when humans spend the day outside in bright daylight, and the nights from sunset to sunrise in the dark. However, many modern workplaces and living environments are characterized by thousand-fold dimmed light during the day (e.g. 50-100 lux inside, as compared to 50,000-100,000 lux outside) and ten thousand-fold brightening during the night (100 lux or more inside as compared to 0.01 lux outside in the pre-electric world).

In such indoor electrical illumination environments, the lack of sufficient light during the day, and excessive light exposure in the evening and night hours in both indoor and outdoor electrical illumination environments leads to disruption of the human circadian timing system and disrupted sleep, depressed mood and ill-health including substantially increased risks of obesity, diabetes, breast cancer and prostate and other neuroendocrine sensitive cancers.

Lighting is designed primarily to provide illumination, and conventional lighting solutions are not developed with the effects on human circadian functioning in mind. Accordingly, suboptimal circadian effects are witnessed, such as undesired circadian stimulation, difficulty maintaining alertness, delays in sleep onset and disrupted sleep, among others. Furthermore, energy efficient LEDs in the market are rich in specific wavelengths that trigger the metabolic processes that disrupt the circadian system and/or melatonin suppression and cause phase shifting in the timing of vital body processes. Artificial lighting is prevalent and if proper consideration is not taken in providing improved adjustments to the lighting, there may be inadvertent harm caused to proper circadian functioning.

Circadian functioning and human performance are related to one another. Lighting mechanisms that are designed with circadian cycles and functioning in mind are therefore desirable and may aid in the addressing physiological challenges that arise in modern workplaces, homes, vehicles, and other indoor environments.

### SUMMARY

The present disclosure generally relates to the field of illumination, and more particularly, apparatus and methods for providing optimal light to maintain healthy and robust human circadian function. Healthy and robust human circadian function can include ensuring alertness and sound cognitive abilities are available when needed, and ensuring that rest cycles are able to promote healing and restoration.

The circadian optimized light of various embodiments is specifically designed in relation to physiologically optimized light spectra in accordance with research conducted by Applicant.

In particular, there is substantial evidence that exposure to specific types of light at various times of a circadian cycle for an individual can have deleterious health effects, such as increasing a risk of obesity and diabetes.

For example, blue-rich light during the day can be protective, but exposure to the same blue rich LED or fluorescent light during the night is harmful. To avoid this risk, improved lighting devices/components, lighting fixtures, methods for producing light, controllers for controlling light output, and corresponding computer-readable media are contemplated. The lighting can be utilized in workspaces, residential and recreational facilities, health care, educational and military facilities, on airplanes, in automobiles, among other applications where circadian functioning is important.

As described in some embodiments, the circadian optimized polychromatic light is a white light, or an substantially white light. The lighting is described for providing improved lighting during various times of day, and is specifically optimized in relation to promoting corresponding physiological effects. A blue-rich light is described in an aspect, as well as a polychromatic white light that can be adapted for day and/or night circadian optimization.

Controlled lighting systems are described in other embodiments designed to track or to establish circadian schedules. Accordingly, in some cases, the physiological effect is protective (e.g., avoiding undesired modification of physiological processes), while in other cases, the physiological effect is remedial (e.g., entrainment or gradual adjustment to a new time zone). The physiological effect of the lighting may be particularly useful for individuals who are adjusting to changes or adapting to non-standard working hours (e.g., evening shift, night shift workers).

The circadian lighting apparatus and/or systems utilize a newly discovered Circadian Potency Spectral Sensitivity Distribution (“Circadian Potency SSD”). The Circadian Potency SSD, and properties of the human circadian system described herein, are used to enable the design and construction of improved lighting systems, controls and sensors which enable optimize human circadian function, performance and health. In some embodiments, the lighting also includes filtering or light injection mechanisms for eyewear, such as eyeglasses, sunglasses, contact lenses, among others. This Circadian Potency SSD is incorporated into the adaption of light sources, and provides critical specifications for designing the Spectral Power Distribution (SPD), which is set of relative irradiance or relative photon flux for each nm interval or bin of wavelengths in the visible light range that is emitted by lighting systems that are adapted for generating emitted light in accordance with the Circadian Potency SSD.

The lighting distribution, in some embodiments, can be controlled by a controller device that sends control signals to one or more lighting devices to modify or otherwise constrain their outputs. In other embodiments, the lighting distribution is provided by way of specially configured lighting elements, such as phosphors, light emitting diodes, etc., whose lighting output, individually or in combination, provides light in accordance with a Circadian Potency Spectral Sensitivity Distribution described herein, for example, as a substantially blue light (e.g., for entrainment), or a substantially white or near-white light (e.g., for office work).

In accordance with some aspects, lighting devices are disclosed which optimize circadian daytime stimulus as defined by the newly disclosed Circadian Potency SSD function which provide peak output at approximately 478 nm and are rich in 440-490 nm light, according to exemplary embodiments. This Circadian Potency SSD function was derived from an initial analysis of human subject data collected by the Applicants. In accordance with Applicants' research, the peak output and Circadian Potency SSD may change based on the age, gender and ethnicity of the participants, as differing circadian effects were noted in empirical study data. Accordingly, there may be some variation in the specific peak or curve distribution to account for human variation, and these variations of 5-10 nm are contemplated.

In accordance with some aspects, based on a subsequent more comprehensive analysis of the data based on a larger subject population and improved methods, lighting devices are disclosed which optimize circadian daytime stimulus as defined by the newly disclosed Circadian Potency SSD function which provide peak output at approximately 472 nm (or in some alternate embodiments at 478 nm) and are rich in 435-485 nm light, according to exemplary embodiments.

In accordance with Applicants' research, the peak output and Circadian Potency SSD may change based on the age or gender or ethnicity or other inter-individual differences of the participants, as differing circadian effects were noted in empirical study data.

In accordance with other aspects, depending on the age, gender, or ethnicity of an individual, lighting devices are disclosed which optimize circadian daytime stimulus as defined by a newly disclosed Circadian Potency SSD functions which provide peak outputs at other values between 470 and 480 nm. For example, the peak could be a wavelength between 470 and 480 nm, such as 471 nm, 472 nm, 473 nm, 474 nm, 475 nm, 476 nm, 477 nm, 478 nm, and 479 nm, for example, in accordance with various embodiments. 472 and 478 nm were found to be particularly optimal but not all embodiments are thus limited.

The Circadian Potency SSD of an embodiment has a skewed peak distribution and an overall “bell” shape. The “bell shape” can be skewed such that a side of the bell shape has a faster rate of attenuation. The skewing, for example, can include a negatively skewed asymmetric distribution around a peak. In a first preferred embodiment, the peak is substantially around 472 nm or at 472 nm. In a second preferred embodiment, the peak is substantially around 478 nm or at 478 nm. This bell-shape can be incorporated into an overall white or near-white light distribution, for example, by adjusting lighting contribution at other wavelength ranges to ensure that the light maintains the overall white or near-white light distribution.

A modifying factor in respect of the peak is based on considerations around practical implementation, for example, effects at a table top level or at an eye level. A number of variant curve types and shapes are contemplated (e.g., a 472 nm peak and 76% in the FW HM between 435-483 nm). In some embodiments, the curve may have non-idealities and accordingly, there may be other sub-peaks or other spectral features that are introduced in a non-ideal curve.

These lights, in some embodiments, utilize specifically configured formulations of phosphors on LED dies based on the Circadian Potency SSD function, or require innovative combinations of monochromatic LED dies, or require innovative combinations of white LEDs and monochromatic LEDs. Lighting technology other than LEDs are also contemplated, for example, specifically tunable lighting, filtered lighting, etc. can be used in accordance with incandescent, fluorescent lighting, bioluminescence lighting, chemi-luminescence lighting, among others.

The circadian lighting apparatus and/or systems can be controllably combined with devices described in Applicants' U.S. application Ser. No. 14/874,601, entitled “Lighting system for protecting circadian neuroendocrine function”, incorporated herein by reference. The lighting systems described in the U.S. application Ser. No. 14/874,601 can be utilized for establishing protective lighting for night usage, which can be toggled or otherwise gradually transitioned between.

The lighting devices are adapted to optimize circadian daytime stimulus as defined by the newly disclosed Circadian Potency SSD function based on the initial analysis, which provides peak output at approximately 472 nm or at 478 nm and are rich in 440-490 nm light, even when the user dims the overall light intensity measured in lux. The lighting devices, for example, can be used in indoor environments where the illuminance leves are reduced to minimize glare and improve comfort but still provide a strong circadian daytime stimulus by increasing the fraction of blue rich light emitted in the total visible spectrum.

In other embodiments, the lighting devices are adapted to optimize circadian daytime stimulus as defined by the newly disclosed Circadian Potency SSD function based on the subsequent more comprehensive analysis, which provides peak output at approximately 472 nm and are rich in 435-485 nm light, even when the user dims the overall light intensity measured in lux. The lighting devices, for example, can be used in indoor environments where the illuminance levels are reduced to minimize glare and improve comfort but still provide a strong circadian daytime stimulus by increasing the fraction of blue rich light emitted in the total visible spectrum.

In accordance with other aspects, depending on the age, gender, or ethnicity of an individual, in other embodiments, lighting devices are disclosed which optimize circadian daytime stimulus as defined by newly disclosed Circadian Potency SSD functions which provide peak outputs at other values between 470 and 480 nm and are rich in 440-490 nm light, even when the user dims the overall light intensity measured in lux. The lighting devices, for example, can be used in indoor environments where the illuminance leves are reduced to minimize glare and improve comfort but still provide a strong circadian daytime stimulus by increasing the fraction of blue rich light emitted in the total visible spectrum.

The lighting devices are adapted to optimize circadian night time protection as defined by the newly disclosed Circadian Potency SSD function based on the initial analysis, which provides minimum spectral output at approximately 478 nm and are depleted in 440-490 nm light. The lighting devices, for example, can be utilized during hours of natural environment darkness from sunset to sunrise by minimizing emission of light wavelengths between 440-490 nm. During these times, the reduction of emission can require the use of unique notch filters to prevent the transmission of 440-490 nm light (among other approaches).

The lighting devices are adapted to optimize circadian night time protection as defined by the newly disclosed Circadian Potency SSD function based on the subsequent more comprehensive analysis, which provide minimum spectral output at approximately 472 nm and are depleted in 435-485 nm light. The lighting devices, for example, can be utilized during hours of natural environment darkness from sunset to sunrise by minimizing emission of light wavelengths between 435-485 nm. During these times, the reduction of emission can require the use of unique notch filters to prevent the transmission of 435-485 nm light (among other approaches).

In accordance with other aspects, depending on the age, gender, or ethnicity of an individual, in other embodiments, lighting devices are disclosed which optimize circadian night time protection as defined by newly disclosed Circadian Potency SSD functions which provide minimum outputs at other values between 470 and 480 nm and are depleted in 440-490 nm light. The lighting devices, for example, can be utilized during hours of natural environment darkness from sunset to sunrise by minimizing emission of light wavelengths between 435-490 nm. During these times, the reduction of emission can require the use of unique notch filters to prevent the transmission of 435-490 nm light (among other approaches).

Control systems are disclosed that are used to transition the spectral power distribution of light sources from maximal circadian stimulation based on the newly disclosed Circadian Potency SSD functions to minimum circadian disruption based on the newly disclosed Circadian Potency SSD functions depending on at least one of the time of day, season of the year, geographical location, presence in illuminated environment, and/or chronobiological characteristics of an individual. In-between or transitionary phases are also contemplated.

The presence, location and/or characteristics of individuals exposed to the light sources may be determined, for example, through the use of wearable devices, facial recognition, security system logs, and expected schedules.

The light devices may be controlled and/or operated through various means and/or systems that may range from fully automated digital systems to analog systems controlling the light sources through the control of power provided to the light sources. In some embodiments, the lights devices may be controlled through the use of one or more control systems, which may send instructions and/or other command signals to the light devices. The one or more control systems may be implemented using, for example, computing devices having non-transitory computer readable media and/or various data interfaces. The one or more control systems may include servers, and may be implemented on various technologies and platforms.

Circadian light sensing apparatus and/or systems are disclosed based on the newly disclosed Circadian Potency SSD function which can accurately assess the circadian stimulus in any polychromatic white light source at the light intensities used in workplaces and homes and other indoor and outdoor environments. The use of these sensing apparatus and/or systems enables the design and construction of improved light sensing systems to ensure lights in a built environment are achieving desired circadian health and performance outcomes for the occupants. These light sensors also enable close-loop control of the out of circadian optimized light fixtures and bulbs.

Circadian Potency conversion factors, waveform descriptions, and algorithms are disclosed which enable the Spectral Power Distribution (SPD) of light sources in a room to be measured at a fixed point in the room (e.g. table top, floor, or wall or ceiling or light fixtures) and to be converted into the active effective SPD and Circadian Potency of the vertical illuminance at the room occupants' eyes. The biologically relevant spectral and intensity characteristics of light is the light entering the room occupant's eyes and the conversion factors enable the Circadian Potency of to be estimated without requiring room occupants to wear or be equipped with wearable or portable devices to record corneal (eye level) light spectral exposure.

While visible light is emitted across a 380 nm-780 nm wavelength range, it is generally accepted that the beneficial effects of light on the circadian timing system during the day and the harmful effects at night are mediated by specialized retinal photoreceptors in the human eye, that are most sensitive to a narrower band of predominantly blue wavelengths.

The relative sensitivity of the human circadian timing system to light at each wavelength in the visible range is defined by a relative circadian spectral sensitivity distribution with the wavelength with the greatest impact on the circadian system being normalized to a value of 1 (Relative Circadian SSD).

Disclosed herein is a previously unknown Relative Circadian SSD for humans living and working in normal electrically illuminated environment occupancy conditions under polychromatic white light (“Circadian Potency SSD”). As described below, previously determined Relative Circadian SSDs were for short, less than 90 minute, duration exposures of dark-adapted eyes to monochromatic light, or were theoretical predictions based on retinal pigment photochemistry and neither of which predict the Circadian Potency SSD disclosed herein in various embodiments.

In an aspect, the light source for daytime applications is configured for emitting light having a spectral power distribution in a visible wavelength range between 380 nm and 700 nm, the spectral power distribution including a circadian optimized peak. The spectral power distribution can include a distribution, or a concentration of a photometric quantity. In a non-limiting, preferred embodiment, the spectral power distribution is defined as one or more proportions of radiant power across various wavelength ranges, for example, as a proportion of the visible wavelength range. In this example, the integrated area under a SPD curve can be utilized to determine a proportion (e.g., a percentage) of light within a particular wavelength range band as a proportion of all radiant power in the visible wavelength range.

In an aspect, the light source for nighttime applications is configured for emitting light having a spectral power distribution in a visible wavelength range between 380 nm and 700 nm, the spectral power distribution including a circadian optimized trough of zero or low emissions. The spectral power distribution can include a distribution, or a concentration of a photometric quantity. In a non-limiting, preferred embodiment, the spectral power distribution is defined as one or more proportions of radiant power across various wavelength ranges, for example, as a proportion of the visible wavelength range. In this example, the integrated area under a SPD curve can be utilized to determine a proportion (e.g., a percentage) of light within a particular wavelength range band as a proportion of all radiant power in the visible wavelength range.

The spectral power distribution of the Circadian Potency SSD can have a negatively skewed asymmetric distribution around the circadian optimized peak, and the circadian optimized peak determined in the initial analysis of the data is at approximately 478 nm (e.g., +/−1, 3, 5, 7 nm). In variant embodiments, the circadian optimized peak is selected from the group of wavelengths consisting of 475, 476, 477, 478, 479, 480, 481, and 482 nm.

In accordance with other aspects, depending on the age, gender, or ethnicity of an individual, The Circadian Potency SSD described can have a negatively skewed asymmetric distribution around the circadian optimized peak, and the circadian optimized peak determined in a subsequent more comprehensive analysis of the data is at approximately 472 nm (e.g., +/−1, 3, 5, 7 nm). In variant embodiments, the circadian optimized peak is selected from the group of wavelengths consisting of 470, 471, 472, 473, 474, 475, 476, 477, 478, 479, and 480 nm.

For a blue-focused light, the negatively skewed asymmetric distribution of the Circadian Potency SSDs described includes significant attenuation in wavelength ranges of the spectral power distribution greater than the circadian optimized peak. In an example embodiment, there is a sharper post-peak reduction of SPD relative to the pre-peak SPD. More specifically, the negatively skewed asymmetric distribution can include gradual attenuation in wavelength ranges of the spectral power distribution less than the circadian optimized peak relative to the significant attenuation in the wavelength ranges of the spectral power distribution greater than the circadian optimized peak.

The negatively skewed asymmetric distribution can be described in various ways, including, for example, as a curve having a mode, a mean, and a median defining a Pearson's co-efficient of skewness, having a specific kurtosis value that defines the shape of the curve. The negatively skewed asymmetric distribution can be a negatively skewed asymmetric normal distribution having a pre-defined standard deviation.

In particular, an improved light is described that is a circadian optimized light.

In a first aspect, the improved light can be a substantially blue light that emits a circadian day optimized light. The circadian day optimized light of this aspect is provided from a light source configured for emitting light having a spectral power distribution in a visible wavelength range between 380 nm and 700 nm, the spectral power distribution including a circadian day optimized peak, the spectral power distribution having a negatively skewed asymmetric distribution around the circadian day optimized peak, the circadian day optimized peak at about 472 nm or at about 478 nm.

In an embodiment, the circadian day optimized peak is at about 472 nm, and 76% of spectral power is emitted within a full width half maximum (FWHM) range of the circadian day optimized peak between about 435 nm to about 483 nm.

In an embodiment, the circadian day optimized peak is at about 478 nm.

In an embodiment, the negatively skewed asymmetric distribution is defined by the relation:

$$P ( λ ) = \frac{1}{σ * √ 2 π} * {e}^{- \frac{{( λ - μ )}^{2}}{2 {σ}^{2}}}$$

wherein σ is a standard deviation, λ is a wavelength, and μ is a mean set at the circadian day optimized peak, and separate values for a are applied at different sides of the negatively skewed asymmetric distribution.

In another embodiment, the negatively skewed asymmetric distribution substantially fits along a curve defined by the relations:

$$P ( λ ) = \frac{1}{33.5 * √ 2 π} * {e}^{- \frac{{( λ - 478.3 )}^{2}}{2 * {33.5}^{2}}} ( left side of the peak )$$

$$P ( λ ) = \frac{1}{8.9 * \sqrt{2 π}} * {e}^{- \frac{{( λ - 478.3 )}^{2}}{2 * {8.9}^{2}}} ( right side of the peak ) .$$

In another embodiment, the negatively skewed asymmetric distribution substantially fits along a curve defined by the relations:

$$P ( λ ) = \frac{1}{3 \; 1.7 * √ 2 π} * {e}^{- \frac{{( λ - 472.2 )}^{2}}{2 * {31.7}^{2}}} ( left side of the peak )$$

$$P ( λ ) = \frac{1}{9.2 * \sqrt{2 π}} * {e}^{- \frac{{( λ - 472.2 )}^{2}}{2 * {31.7}^{2}}} ( right side of the peak ) .$$

In an embodiment, the left side of the negatively skewed asymmetric distribution has a standard deviation (σ) value of 30, 31, 32, 33, 34, or 35.

In an embodiment, the right side of the negatively skewed asymmetric distribution has a standard deviation (σ) value of 7, 8, 9, 10, or 11.

In an embodiment, the negatively skewed asymmetric distribution substantially fits along the curve having a tolerance defined by an R-squared value of the regression line between circadian potency/photopic power ratio and melatonin AUC greater than at least one of 0.90, 0.95, 0.96, 0.97, 0.98, or 0.99.

In an embodiment, the negatively skewed asymmetric distribution includes at least 78% of an area under a curve defined by the spectral power distribution in the visual wavelength range between about 440 and about 490 nm.

In an embodiment, the negatively skewed asymmetric distribution is a mirror image log-normal distribution.

In an embodiment, the negatively skewed asymmetric distribution is comprised of, or consists of, a first half-Gaussian function and a second half-Gaussian function.

In an embodiment, the circadian day optimized light is substantially blue, and only includes light in a wavelength range between approximately 380 nm and approximately 505 nm.

Polychromatic light is made up of several colors, which combine to generate an overall light based on the combination of the constituent lighting. Accordingly, polychromatic light includes radiation of more than one wavelength and has a spectral distribution profile. A polychromatic light can be established using combinations of monochromatic light (e.g., a combination of blue, red, green, or cyan, magenta, yellow, black, among others).

In an embodiment, the spectral power distribution further includes supplemental polychromatic light provided in the 480-700 nm wavelength range, the supplemental polychromatic light adapted such that the light provided by the light emitting device is substantially white or near-white.

In a second aspect, a light emitting device for emitting a circadian day optimized polychromatic white or near white light is described, the light emitting device including a light source configured for emitting light having a spectral power distribution in a visible wavelength range between 380 nm and 780 nm, the spectral power distribution including a circadian day optimized peak, and the spectral power distribution further includes greater than approximately 20% of total 380-780 nm visible irradiance in the 440-490 nm wavelength band.

In an embodiment, the polychromatic white or near white light is provided within an oval space on the CIE 1931 chromaticity diagram with the coordinates of the long axis between x=0.47, y=0.45 and x=0.21, y=0.26, and coordinates of the short axis between x=0.31, y=0.40 and x=0.37, y=0.30.

In an embodiment, the polychromatic white or near white light is provided defined according to the ANSI standard C78.377-2008 using the Planckian locus (or black body line) where the white light area for the range of CCTs between 2700K to 6500K is described by the length of the white CCT lines which are each 0.012 Duv in length (and are +−0.006 Duv from the Planckian locus).

In an embodiment, the circadian day optimized peak is selected from the group of wavelengths consisting of 467, 468, 469, 470, 471, 472, 473, 474, 475, 476, 477 nm.

In an embodiment, the circadian optimized peak is selected from the group of wavelengths consisting of 480, 481, 482, 483, 484, 485, 486, 487, 488, and 489 nm when illuminated environment occupants include at least one occupant whose age is greater than 50 years of age.

In an embodiment, the spectral power distribution includes an absolute maxima at about 472 nm or at about 478 nm, and a relative minima at about 500 nm.

In an embodiment, the spectral power distribution includes a relative maxima at about 600, 605, 610, or 620 nm.

In an embodiment, the spectral power distribution between about 480 nm to about 700 nm balances with a power distribution around the circadian day optimized peak to provide an optimized polychromatic white light.

In an embodiment, the spectral power distribution between about 400 nm to 480 nm approximates a along a curve defined by the relations:

$$P ( λ ) = \frac{1}{33.5 * √ 2 π} * {e}^{- \frac{{( λ - 478.3 )}^{2}}{2 * {33.5}^{2}}} ( left side of the peak ) and$$

$$P ( λ ) = \frac{1}{8.9 * \sqrt{2 π}} * {e}^{- \frac{{( λ - 478.3 )}^{2}}{2 * {8.9}^{2}}} ( right side of the peak ) .$$

In an embodiment, the spectral power distribution between about 400 nm to 480 nm approximates a along a curve defined by the relations:

$$P ( λ ) = \frac{1}{31.7 * √ 2 π} * {e}^{- \frac{{( λ - 472.2 )}^{2}}{2 * {31.7}^{2}}} ( left side of the peak ) and$$

$$P ( λ ) = \frac{1}{9.2 * \sqrt{2 π}} * {e}^{- \frac{{( λ - 472.2 )}^{2}}{2 * {9.2}^{2}}} ( right side of the peak ) .$$

In an embodiment, the light source includes a first white light emitting diode including a 450 nm blue pump die coated with phosphors, and a second monochromatic blue light emitting diode having a peak emission in a range of about 475-480 nm.

In an embodiment, the second monochromatic blue light emitting diode produces light that is adapted to fill in troughs in an emission of the first white light emitting diode in a 478 nm wavelength region.

In an embodiment, the light source includes a light emitting diode comprising a die with a peak emission at 478 nm which is coated with phosphors adapted to provide a strong photopic emission between 500 nm and 700 nm.

In an embodiment, the light source includes an array of red, green and blue light emitting diode chips where the blue light emitting diode has a peak emission at approximately 475-480 nm, which, in combination, are powered to create the circadian-optimized polychromatic light that is substantially white.

In a third aspect, a light emitting device is provided for emitting a circadian night optimized polychromatic white light with a color within an oval space on the CIE 1931 chromaticity diagram with the coordinates of the long axis between x=0.47, y=0.45 and x=0.21, y=0.26, and coordinates of the short axis between x=0.31, y=0.40 and x=0.37, y=0.30. The light emitting device includes a light source configured for emitting light having a spectral power distribution in a visible wavelength range between 380 nm and 700 nm, the spectral power distribution including a circadian night optimized trough, the spectral power distribution having less than 2% of total 380-780 nm visible irradiance in the 440-490 nm wavelength band.

In an embodiment, the circadian night optimized trough is selected from the group of wavelengths consisting of 467, 468, 469, 470, 471, 472, 473, 474, 475, 476, 477 nm.

In an embodiment, the circadian night optimized trough is selected from the group of wavelengths consisting of 473, 474, 475, 476, 477, 478, 479, 480, 481, 482 and 483 nm.

In an embodiment, the circadian night optimized trough is selected from the group of wavelengths consisting of 480, 481, 482, 483, 484, 485, 486, 487, 488, and 489 nm when illuminated environment occupants include at least one occupant whose age is greater than 50 years of age.

In an embodiment, the light emitting device including a violet light emitter which, in combination with the spectral power distribution in the visible wavelength range between 380 nm and 700 nm including the circadian night optimized trough causes the circadian-optimized polychromatic light that to be substantially white.

In an embodiment, the circadian night optimized trough is approximately an inverse of a negatively skewed asymmetric distribution around a circadian day optimized peak.

The light emitting devices of some embodiments can be used for improving circadian potency of the light as applied to human circadian functioning of individuals exposed to the light, or for causing circadian stimulation of individuals exposed to the light, including for circadian entrainment.

In a fourth aspect, a lighting system is proposed for providing either circadian day optimized polychromatic white light or circadian night optimized polychromatic white light including: a first light emitting device for emitting the circadian day optimized polychromatic white light, and a second light emitting device for emitting the circadian night optimized polychromatic white light, according to various embodiments.

In a fifth aspect, a lighting system is proposed for providing either circadian day optimized polychromatic white light or circadian night optimized polychromatic white light including: a light emitting device configured for at least a first mode of operation and a second mode of operation, the light emitting device in the first mode configured for emitting the circadian day optimized polychromatic white light, the light emitting device in the second mode configured for emitting the circadian night optimized polychromatic white light, according to various embodiments.

In an embodiment, a controller circuit is configured to provide electronic signals controlling the light emitting device to toggle operation between the first mode of operation and the second mode of operation. The controller circuit can be configured to toggle operation between the first mode of operation and the second mode of operation in accordance with a circadian schedule adapted for one or more users exposed to the light from the lighting system, or to entrain the circadian schedule of the one or more users.

For example, the lighting system can be provided on a vehicle travelling from an origin region to a destination region, and the circadian schedule is adapted to entrain the circadian functioning of the one or more users exposed to the light from the lighting system based on a time zone of one of the origin region and the destination region

Other embodiments are contemplated in relation to the use of a circadian day optimized light (CDay) in conjunction with a circadian night (CNight) optimized light, which may be selectively activated depending on a type of desired circadian effect (e.g., stimulus or protection).

Dimmer variations are also contemplated whereby the overall amount of power in the light is adjusted or controlled in relation to a dimming effect being generated by the control of the light. For example, during a time when circadian stimulation is desired, although the CDay light is being dimmed, to maintain effective power at the circadian optimized peak, the circadian potency is managed and the SPD of the light may be adjusted to account for the increased kurtosis of the circadian optimized peak (e.g., to deliver a consistent amount of irradiance despite the dimming).

The circadian night optimized light can include one or more violet light emitting devices (e.g., phosphors, light emitting diodes, violet pumps) that increase an intensity of light at violet wavelength ranges of approximately 410 nm to approximately 430 nm to compensate for a reduction or attenuation of light in accordance with the Circadian Potency SSD (e.g., a reduction in light at night where light under the Circadian Potency SSD SPD curve is removed or otherwise attenuated). Without compensation, the light may appear unduly orange or yellow. Accordingly, a substantially white light for the night circadian functioning optimized polychromatic light can be provided despite the reduction or attenuation of light in accordance with the circadian-day optimized spectral power distribution.

Controller circuits can be utilized to control the transition of lighting from the circadian night optimized light and the circadian day optimized light. In some embodiments, a single combined lighting is provided that is tunable between spectral profiles associated with the circadian night optimized light and the circadian day optimized light. Tunability can include the use of filters, tunable phosphor designs, light emitting devices having different lighting profiles responsive to different voltage/power inputs, among others.

In other embodiments, a lighting system is provided that has separate circadian night optimized lights and the circadian day optimized lights, and a controller circuit is utilized to transition between the use of the separate circadian night optimized lights and the circadian day optimized lights. Transition can be on/off for each, or may be a controlled mix of power provided to each (e.g., gradual dimming/powering up). The control may be established based on circadian states being tracked (either desired circadian states for entrainment or maintenance of existing circadian states).

In another embodiment, the spectral power distribution for circadian functioning is tracked by a configured light measuring device, which tracks a quantity of incident light having a spectral power distribution in a visible wavelength range between 380 nm and 700 nm to detect the quantity of incident light that is provided within the Circadian Potency SSD defined stimulatory wavelengths for circadian functioning. The light measuring device can be specifically attuned for tracking the incident light, for example, having specific sets of filters and photosensors/photodetectors that are developed such that the light measuring device captures light measurements within the spectral power distribution for circadian functioning as described herein.

The light measurement device in some embodiments may incorporate and utilize Circadian Potency conversion factor algorithms which enable the Spectral Power Distribution (SPD) of light sources in a room to be measured at a fixed point in the room (e.g. table top, floor, or wall or ceiling or light fixtures) and to be converted into the active effective SPD and Circadian Potency of the vertical illuminance at the room occupant's eyes. The biologically relevant spectral and intensity characteristics of light is the light entering the room occupant's eyes and the conversion factors enable the Circadian Potency of the lighting environment to be estimated without requiring room occupants to use wearable devices to record light spectral exposure.

A controller circuit may receive the light measurements in the form of electronic signals, and in some embodiments, the controller circuit may capture readings in wavelength designated “bins” (e.g., specific ranges), which are then associated with different weighting based on the spectral power distribution for circadian functioning as described herein. The combined effect on circadian functioning can be determined based on the weighted effect from the various bins.

In some embodiments the controller circuit may incorporate and utilize Circadian Potency conversion factors and algorithms which enable the Spectral Power Distribution (SPD) of light sources in a room to be measured at a fixed point in the room (e.g. table top, floor, or wall or ceiling or light fixtures) and to be converted into the active effective SPD and Circadian Potency of the vertical illuminance at the room occupants' eyes. The biologically relevant spectral and intensity characteristics of light is the light entering the room occupant's eyes and the conversion factors enable the Circadian Potency can thereby be estimated without requiring room occupants to use wearable devices to record light spectral exposure.

The conversion factors can be used with corresponding sensors, which measure the SPD of light sources at the room at the eye level, which can be used as a baseline for converting the light using the conversion factors. The conversion of the light can be translated into lighting control signals (e.g., data packets specifying desired illumination at specific wavelengths) that modify the output of the lighting devices, such that the output, as it reaches the eye level (including environmental effects due to the room) is in accordance with a desired Spectral Power Distribution (SPD), such as the Circadian Potency SSD adapted for the day or the Circadian Potency SSD adapted for the night.

Corresponding methods are contemplated, including, but not limited to, in a first aspect, a method for emitting light having a spectral power distribution in a visible wavelength range between 380 nm and 700 nm, the spectral power distribution including a circadian day optimized peak, the spectral power distribution having a negatively skewed asymmetric distribution around the circadian day optimized peak, the circadian day optimized peak at a wavelength between about 472 nm to about 478 nm; in a second aspect, a method for emitting a circadian day optimized polychromatic white or near white light having a spectral power distribution in a visible wavelength range between 380 nm and 780 nm, the spectral power distribution including a circadian day optimized peak; and wherein the spectral power distribution further includes greater than approximately 20% of total 380-780 nm visible irradiance in the 440-490 nm wavelength band; and in a third aspect, a method for emitting a circadian night optimized polychromatic white light with a color within an oval space on the CIE 1931 chromaticity diagram with the coordinates of the long axis between x=0.47, y=0.45 and x=0.21, y=0.26, and coordinates of the short axis between x=0.31, y=0.40 and x=0.37, y=0.30, by emitting light having a spectral power distribution in a visible wavelength range between 380 nm and 700 nm, the spectral power distribution including a circadian night optimized trough, the spectral power distribution having less than 2% of total 380-780 nm visible irradiance in the 440-490 nm wavelength band.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is a CIE 1931 color space chromaticity diagram indicating areas attributed to white light and to other distinct colors 1931 (adopted from Gage et al., 1977). The white light region falls within the dashed oval shape on the CIE 1931 diagram.

FIG. 2 illustrates one example of an indoor environment, having artificial lighting provided by recessed ceiling troffer panels, pendant fixtures, downlights, highbays, desk and task lights and wall sconces, according to some embodiments.

FIG. 3 includes FIG. 3A and FIG. 3B; A) is a diagram illustrating the apparatus used by Brainard et al (2001) to test the physiological responses to monochromatic light sources in dark adapted subjects with pharmacologically diluted pupils, B) shows the short-term response of melatonin to these light sources measured over a 90 minute light exposure.

FIG. 4 includes FIG. 4A and FIG. 4B, which compares A) the spectral sensitivity curve derived by Thapan et al (2001) with 30 minute light treatments and the B) the spectral sensitivity curve derived by Brainard et al with 90 minute light treatments.

FIG. 5 shows the circadian stimulus (CS) model (Figuero, 2017) spectral sensitivity curves for “warm” and “cool” Coordinate Color Temperature (CCT) lights.

FIG. 6A, 6B, 6C shows the data published by Gooley et al (2010) for six-hour exposures to either blue (460 nm) light or green (555 nm) light. A) the spectra of the two (blue and green) light sources. B) the suppression of melatonin time course with blue light (top) and with green light (bottom) six-hour exposures as compared to control melatonin levels measured in darkness. C) the time course of the decay in response to green light indicating it had a transient effect as compared to blue light.

FIG. 7 shows the distribution of work shift lengths in a survey of 224 companies with day, evening and night work shifts from Shiftwork Practices 2017.

FIG. 8 is an illustration of an Equivalent Melanopic Lux spectral sensitivity curve.

FIG. 9 illustrates a contrast between the spectral sensitivity curves of two leading models used in the lighting industry, the Equivalent Melanopic Lux (EML) and the Circadian Stimulus (CS).

FIG. 10 shows the results of a survey of lighting specifiers and designers showing the relative usage of EML and CS models Edward Clark and Natalia Lesniak Circadian Lighting Solutions Are Real and Important—Why Aren't They Being Used? Metropolis Magazine Architectural/Designer Survey December 2017 http://www.metropolismag.com/design/circadian-lighting-survey/.

FIG. 11 is a graph of relative irradiance plotted against wavelength, showing the spectral power distribution of seven different light sources (labelled A-G) all of which emitted white light within the CIE 1931 white light color space at a table top light intensity of 50 foot candles (540 lux) to assess the impact on spectral wavelength components on human circadian function and melatonin suppression in the initial analysis.

FIG. 12 is a plot of melatonin levels against time of day, illustrating a comparison of mean and standard error of the salivary melatonin levels across a 12 hour overnight light exposure from 20:00 hours until 08:00 hours under two of the light sources (A and G) showing substantial differences in the Melatonin Area Under the Curve (AUC) because of the significantly greater suppression by light source A as compared to light source G.

FIG. 13 is a table illustrating the results of overnight 540 lux light exposure for each of the seven light sources in the initial analysis expressed as mean and standard error of Melatonin AUC.

FIG. 14 is a graph of corneal (eye level) vertical relative irradiance plotted against wavelength, showing the spectral power distribution of seven different light sources (labelled A-G) all of which emitted white light within the CIE 1931 color space at a table top light intensity of 50 foot candles (540 lux) and a corneal light intensity of 250-270 lux to assess the impact on spectral wavelength components on human circadian function and melatonin suppression in the subsequent more comprehensive analysis.

FIG. 15 is a graph of the mean and SEM of the salivary melatonin levels for a larger sample of 73 human subject (47 male/26 female aged 20-41) nights of exposure to 12 hours of white light from the seven different light source SPDs at the same light intensity.

FIG. 16 is a table illustrating the results of overnight 250-270 lux corneal vertical illuminance exposure for each of the seven light sources in the subsequent more comprehensive analysis expressed as mean and standard error of Melatonin AUC determined by the trapezoid method.

FIG. 17 is a plot of relative photopic spectral sensitivity against wavelength, showing the standard photopic power function used to define and measure lux levels.

FIG. 18 provides a diagrammatic representation of the parameters used in the optimization of the best fit relative circadian spectral sensitivity curve for Circadian Potency (P) to the data collected in the study with eight different light sources. The optimization routine independently determined the wavelength of maximum circadian sensitivity (μ), the Gaussian standard deviation (σ) for wavelengths below μ, the Gaussian standard deviation (σ) for wavelengths above μ and thereby determined the short wavelength (left) and long wavelength (right) half maximum widths. The general form of the Gaussian function used in the optimization is provided alongside the function.

FIG. 19 is an illustration of a plot showing the parameters used in the determination of the Circadian Potency/Photopic Power ratio (CPPPR) for each light source. The “Circadian” function is a simplified example for purposes of illustration and does not represent the derived Circadian Potency curves disclosed in this application.

FIG. 20 is a plot of a linear regression best fit optimization from the initial analysis to the CPPPR values for each light source plotted against the Melatonin AUC.

FIG. 21 illustrates an optimized best fit Circadian Potency spectral sensitivity curve determined in the initial analysis with a peak sensitivity at 478 nm derived for healthy human subjects exposed to twelve hours of light overnight at 540 lux table top from multiple white light sources, according to some embodiments.

FIG. 22 is a plot of a linear regression best fit optimization from the subsequent more comprehensive analysis to the CPPPR values derived from the relative corneal SPD from each light source plotted against the Melatonin AUC determined by the trapezoid method.

FIG. 23 illustrates an optimized best fit Circadian Potency spectral sensitivity curve determined in the subsequent more comprehensive analysis with a peak sensitivity at 472 nm derived for healthy human subjects exposed to twelve hours of light overnight at 250-270 lux vertical corneal illuminance from multiple white light sources, according to some embodiments.

FIG. 24 illustrates the initial analysis of the asymmetric Full Width Half Maximum (FWHM) of the Circadian Potency curve with 78.4% of total Circadian Potency falling between 440-490 nm, according to some embodiments.

FIG. 25 illustrates the subsequent more comprehensive analysis of the asymmetric Full Width Half Maximum (FWHM) of the Circadian Potency curve with 76% of total Circadian Potency falling between 435-483 nm, according to some embodiments.

FIG. 26 is a plot of relative circadian spectral sensitivity plotted against wavelength, illustrating a comparison of the Circadian Potency Curve from the initial analysis with the Circadian Stimulus (CS) for cool light above approximately 3300K CCT, according to some embodiments.

FIG. 27 a plot of relative circadian spectral sensitivity plotted against wavelength, illustrating a comparison of the Circadian Potency Curve from the initial analysis with the Circadian Stimulus (CS) for warm light below approximately 3200K CCT, according to some embodiments.

FIG. 28 a plot of relative circadian spectral sensitivity plotted against wavelength, illustrating a comparison of the Circadian Potency Curve with the Equivalent Melanopic Lux (EML) spectral sensitivity curve, according to some embodiments.

FIG. 29 a plot of relative circadian spectral sensitivity plotted against wavelength, showing a comparison of the three parameter best fit curves to the data of Thapan et al (2001) for 30 minutes of light and Brainard et al (2001) for 90 minutes of light.

FIG. 30 a plot of relative circadian spectral sensitivity plotted against wavelength, showing a comparison of the three parameter best fit curves to the data of Thapan et al for 30 minutes of light, Brainard for 90 minutes of light and for the Circadian Potency function derived from the initial analysis based on 12 hours of light exposure with the maximum relative circadian sensitivity for each curve normalized to the value of 1, according to some embodiments.

FIG. 31 a plot of relative circadian spectral sensitivity plotted against wavelength, showing a comparison of the three parameter best fit curves to the data of Thapan et al for 30 minutes of light, Brainard for 90 minutes of light and for the Circadian Potency function derived from the subsequent more comprehensive analysis based on 12 hours of light exposure with the maximum relative circadian sensitivity for each curve normalized to the value of 1, according to some embodiments.

FIG. 32 a plot of normalized circadian spectral sensitivity plotted against wavelength, showing a comparison of the three parameter best fit curves to the data of Thapan et al for 30 minutes of light, Brainard for 90 minutes of light and for the Circadian Potency function from the initial analysis based on 12 hours of light exposure with the relative circadian sensitivity at 478 nm for each curve normalized to the value of 1, according to some embodiments.

FIG. 33 a plot of normalized circadian spectral sensitivity plotted against wavelength, showing a comparison of the three parameter best fit curves to the data of Thapan et al for 30 minutes of light, Brainard for 90 minutes of light and for the Circadian Potency function from the more comprehensive subsequent analysis based on 12 hours of light exposure with the relative circadian sensitivity at 472 nm for each curve normalized to the value of 1, according to some embodiments.

FIG. 34 shows the spectral power distribution (SPD) of a light source with an optimal circadian stimulus for day time light which mirrors the Circadian Potency SSD. This light source is blue in color and optimized solely for circadian stimulatory effectiveness.

FIG. 35 shows the location on the CIE 1931 chromaticity diagram of the blue light SPD from FIG. 34 which solely optimizes circadian stimulatory effectiveness.

FIG. 36 shows that other colors must be added to the blue light from the green, yellow and red spectrum indicated by the bar on the right side of the CIE 1931 chromaticity diagram in order to create polychromatic white light that falls within the dashed oval white light space.

FIG. 37 shows the Equivalent Melanopic Lux (EML) (left), Circadian Stimulus (CS) (Cool) (middle) and Circadian Potency SSD (right) plots and a table indicating the available wavelengths for nocturnal light spectral engineering with less than 20% of maximum circadian sensitivity according to the Circadian Potency and EML and CS models, according to some embodiments.

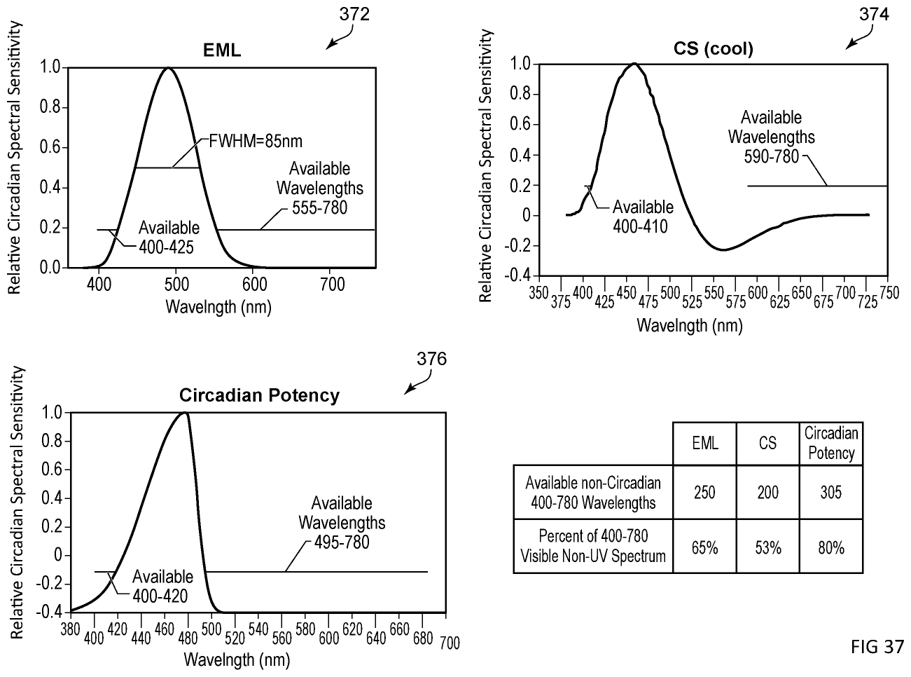

FIG. 38 is a plot of relative irradiance plotted against wavelength, showing a comparison between the spectral power distribution of a conventional blue pump LED with a 4100 CCT and the optimized Circadian Potency function determined in the initial analysis, according to some embodiments.

FIG. 39 is a plot of relative irradiance plotted against wavelength, showing a comparison between the optimized Circadian Potency function with a peak at 478 nm derived in the initial analysis and the spectral power distribution (SPD) of an optimized white LED designed for night use to deliver minimum Circadian Potency as described in various embodiments of this disclosure.

FIG. 40 is a plot of relative irradiance plotted against wavelength, showing a comparison between the optimized Circadian Potency function with a peak at 472 nm derived in the subsequent more comprehensive analysis and the spectral power distribution of an optimized white LED designed for night use to deliver minimum Circadian Potency as described in various embodiments of this disclosure.

FIG. 41 is a plot of relative irradiance plotted against wavelength, showing comparison between the optimized Circadian Potency function with a peak at 472 nm as derived in the subsequent more comprehensive analysis and the spectral power distribution of an optimized white LED designed for daytime use to deliver maximum Circadian Potency as described in this disclosure, according to some embodiments.

FIG. 42 is a plot of relative irradiance plotted against wavelength, showing comparison between the optimized Circadian Potency function with a peak at 478 nm as derived in the initial analysis and the spectral power distribution of an optimized white LED designed for daytime use to deliver maximum Circadian Potency as described in this disclosure, according to some embodiments.

FIG. 43 is a plot of relative irradiance plotted against wavelength, showing comparison between the optimized Circadian Potency function with a peak at 472 nm and the spectral power distribution of an optimized white LED designed for daytime use which has a highest peak emission at or near 472 nm to deliver maximum Circadian Potency as described in this disclosure, according to some embodiments.

FIG. 44 shows three plots of relative irradiance plotted against wavelength and a table, illustrating a comparison of the Percent Blue^{440-490 nm} Irradiance, Relative Circadian Potency, and Circadian Potency/Photopic Power ratio (CPPPR) of the Light sources depicted in FIGS. 38, 39 and 42.

FIG. 45 shows two plots of relative irradiance plotted against wavelength, showing a comparison between the optimized Circadian Potency function with a peak at 478 nm derived in the initial analysis and the spectral power distribution of an optimized white LED designed to deliver maximum Circadian Potency described in this disclosure and, which as the light is dimmed to 50% maintains Circadian Potency, according to some embodiments.

FIG. 46 shows two plots of relative irradiance plotted against wavelength, showing a comparison between the optimized Circadian Potency function with a peak at 472 nm derived in the subsequent more comprehensive analysis and the spectral power distribution of an optimized white LED designed to deliver maximum Circadian Potency described in this disclosure and, which as the light is dimmed to 50% maintains Circadian Potency, according to some embodiments

FIG. 47 depicts example circuitry that maintains constant blue irradiance. One current source (I_{blue}) maintains a constant current through the blue LEDs while the current through the white LEDs is regulated with a second current source (I_{white}).

FIG. 48 depicts an example system that uses a constant-current LED driver and a current-regulator diode. The system current is regulated by the LED driver. The current through the blue LEDs is regulated by the diode. The current through the white LEDs is the difference of the system current and the blue LED current which is I_{white}=I_{system}−I_{blue}

FIG. 49 depicts example circuitry that maintains blue irradiance proportional to the white light output. In a simple design, the current through the blue LEDs is set to a value that is a fixed percentage of the current through the white LEDs (i.e. I_{blue}=K·I_{white} where K is constant.)

FIG. 50 depicts circuitry that uses a current sink and current shunt to control the current through the blue LEDs by sensing the current through the white LEDs and setting the blue LED current to a proportional value

FIG. 51 is a graph showing an example of a conversion factor for converting the absolute irradiance spectral power distribution from a light source measured at table top horizontal illuminance into the absolute irradiance SPD of vertical illuminance at corneal eye level of a seated human occupant in the space. The conversion factor for each room or occupant workstation will be unique depending on the reflectance, absorption and fluorescent qualities of the walls and other surfaces in the room.

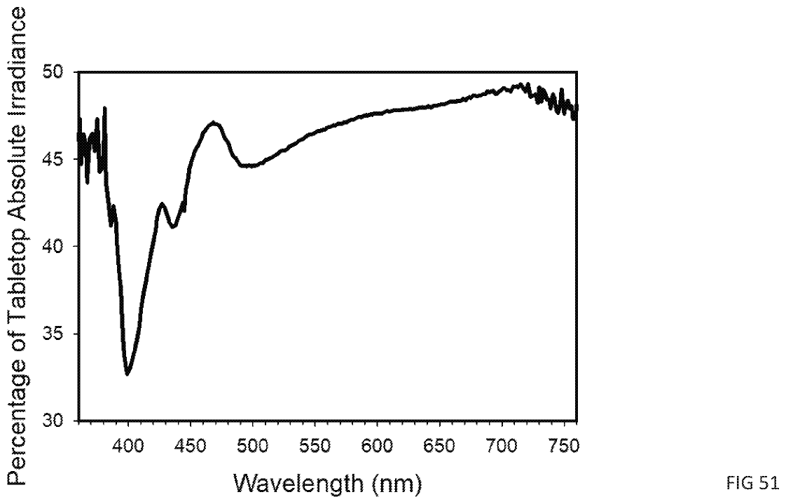

FIG. 52 is an example block diagram of an example lighting circuit controller, according to some embodiments.

### DETAILED DESCRIPTION

Methods and apparatus for circadian lighting are disclosed. In particular, an improved light is described that is a circadian optimized light.

In a first aspect, the improved light can be a substantially blue light that emits a circadian day optimized light. In a second aspect, a light emitting device for emitting a circadian day optimized polychromatic white or near white light is described. In a third aspect, a light emitting device is provided for emitting a circadian night optimized polychromatic white light is described.

These lights utilize newly discovered unanticipated properties of the human circadian system when people are exposed to polychromatic white light. In accordance with some embodiments, the definition of the optimum relative circadian spectral sensitivity distribution (“Circadian Potency SSD”) is established, with a peak sensitivity at approximately 472 nm with a full width at half maximum (“FWHM”) of approximately 48 nm which is skewed asymmetrically between approximately 435 nm and approximately 483 nm. Another relative circadian spectral sensitivity distribution (“Circadian Potency SSD”) is also disclosed based on an initial analysis of a subset of the data with a peak sensitivity at approximately 478 nm with a full width at half maximum (“FWHM”) of approximately 50 nm which is skewed asymmetrically between approximately 440 nm and approximately 490 nm.

A distribution is described herein that includes a circadian day optimized peak, which can be incorporated as a substantially blue light, a circadian day optimized polychromatic white or near white light, or a light emitting device is provided for emitting a circadian night optimized polychromatic white light. For the circadian night optimized polychromatic white light, a circadian night optimized trough is described. The circadian day optimized peak can include a peak at approximately 472 nm or at approximately 478 nm, or other values in the proximity, according to various embodiments. In further embodiments, characteristics of a distribution are described around the circadian day optimized peak.

The circadian night optimized trough can, in some embodiments, be similar or substantially identical to an inverse variation of the circadian day optimized peak and/or the distribution thereof.

As described in further embodiments, the lighting may also be controllable such that overall white or near white light is provided in accordance with a desired or an existing circadian schedule, alternating operation of a lighting system such that the correct lighting is provided for a given time. The lighting system can include separate components which are switched on/off/dimmed in accordance with the schedule, or, in other embodiments, emitters which are tuned in accordance with the schedule. The control may be established through the use of a controller circuit, which sends electronic signals to modify characteristics of the lighting (e.g., data packets, on/off signals). The lighting can also be controllable to tune aspects of the lighting provided, for example, to modify the overall color produced (to ensure that it is white or near white), or to modify characteristics such as warmness, color temperature, overall luminance, etc.

In some further embodiments, converter circuits are also utilized in conjunction with sensors which track a lighting at a position where it is similar to that delivered to an eye of an observer (e.g., someone who is exposed to the light). The lighting actually delivered may be modified, for example, by environmental effects, such as the bouncing of light off of surfaces, non-idealities in light generation, among others, and the conversion circuit may generate conversion signals based at least on the sensed lighting characteristics that modify how lighting is provided by the lighting sources. A feedback loop may be utilized to ensure that the lighting overall tracks changes in the environmental characteristics over a period of time. White light is desirable in some embodiments as is used in workplaces. It may be desirable to switch between two operating modes of light (day and night) without transitioning to another color during the switch, such that people exposed to the light are not or are minimally aware of the shift (e.g., night workers) as not to disturb their work.

Circadian Potency SSD

This disclosure describes methods to determine the Circadian Potency of light sources from their spectral power distributions (SPD) and use the Circadian Potency SSD to optimize healthy circadian function under normal indoor and outdoor illumination conditions. This determination is utilized to modify how lighting is controlled in respect of light output and delivery (e.g., with tunable levels of output, dimmers, modified spectral properties, activation/deactivation of various phosphors, filters, chemical and/or optical properties).

White light sources for daytime use which implement the Circadian Potency SSD to create SPDs with peak spectral power at the peak Circadian Potency wavelengths are described. White light sources for use at night are described which use the Circadian Potency SSD to emit SPDs which minimize Circadian Potency between sunset and sunrise. In addition, systems, controls and sensors are described which use the Circadian Potency SSD to monitor and measure Circadian Potency and maximize Circadian Potency during daytime even when the lights are dimmed by the user, and then minimize Circadian Potency between the hours of sunset and sunrise in the geographic location of the user in order to minimize circadian disruption and improve human health and performance.

The potency of any light entering the eye for causing beneficial circadian synchronizing effects during the day and/or harmful circadian disruptive effects during the night (“Circadian Potency”) can be quantified by the relative spectral power distribution (Relative SPD³⁸⁰⁻⁷⁸⁰) of the light multiplied by the total visible irradiance between 380-780 nm (Irradiance³⁸⁰⁻⁷⁸⁰) multiplied by the Circadian Potency SSD.

Circadian Potency is a property of the light entering the eye or detected by a photometric measurement device. At any given Circadian Potency level certain biological systems or measured physiological variables will be more sensitive and others will be less sensitive, and the response to a light of a given Circadian Potency will also vary depending on time of day, season of the year, geographical variation, prior light exposure and a wide range of biological and biomedical parameters, including but not limited to an individual's genetics, age, health and disease processes.

Since both Relative SPD³⁸⁰⁻⁷⁸⁰ and Circadian Potency SSD are dimensionless, the Circadian Potency of light may be defined in radiometric units of irradiance (such as μW/cm²) which measure the power of electromagnetic radiation incident per unit area on a surface.

Alternatively, Circadian Potency may be defined using quantum units of photon flux (such as photons/cm²/sec) which measure the number of photons per second incident per unit area on a surface. Thus, Circadian Potency can be quantified by multiplying the spectral photon flux density distribution across the 380-780 nm visible wavelength range (SDD³⁸⁰⁻⁷⁸⁰) of the light multiplied by the total visible photon flux between 380-780 nm (Photon Flux³⁸⁰⁻⁷⁸⁰) multiplied by the Circadian Potency SSD.

Irradiance³⁸⁰⁻⁷⁸⁰, Relative SPD³⁸⁰⁻⁷⁸⁰, Photon Flux³⁸⁰⁻⁷⁸⁰, and Relative SSD³⁸⁰⁻⁷⁸⁰ are all well-defined standardized metrics readily measured by standard photometric devices. The only variable which until this present disclosure had not been defined is the Circadian Potency SSD, which is critical for determining the Circadian Potency of any light, and for the design and construction of measurement devices using Circadian Potency SSD.

A fundamental problem for the science and application of circadian optimized lighting and the measurement of the Circadian Potency of any lighting environment is that there is no general consensus on the precise characteristics of the Relative Circadian SSD, with different models, curve shapes and peak sensitivity wavelengths being proposed, and the lack of empirical data from humans living and working in normal electrically illuminated environment occupancy conditions under polychromatic white light.

This has led to the design, manufacture and installation of lights that emit Relative SPD³⁸⁰⁻⁷⁸⁰ which are not beneficial to human health and well-being, and in many cases may harm the individual and cause ill-health. The consequences of selecting and using the wrong Relative Circadian SSD which does not accurately predict human health and performance impacts can be substantial.

Using the wrong Relative Circadian SSD model can also result in the design, manufacture and installation of lights with aesthetically, visual perceptual and functionally undesirable qualities, because unneeded restrictions were placed on designing the SPD of the light.

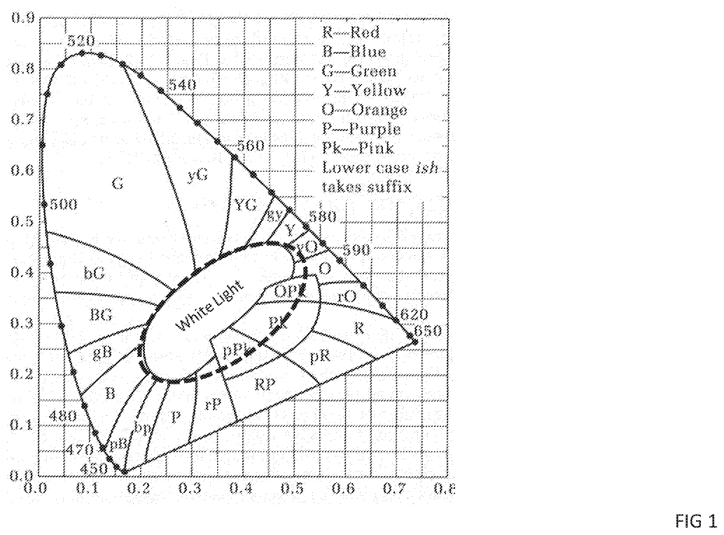

In various embodiments, a novel Relative Circadian SSD is implemented for typical white indoor electric lighting empirically determined using human subjects illuminated by Polychromatic White Light with the Relative SPDs³⁸⁰⁻⁷⁸⁰. Aspects of the lighting, including but not limited to the light intensities, the light sources and light fixtures and the exposure durations to which people are typically exposed can be managed in accordance with the Circadian Potency SSD as described in various embodiments. Polychromatic White Light is defined as light with chromaticity x and y coordinates falling in the area labeled “white” in the CIE 1931 chromaticity diagram in FIG. 1. This white light area falls within an oval shape with the long axis coordinates between x=0.52, y=0.44 and x=0.20, y=0.22, and the short axis coordinates between x=0.42, y=0.24 and x=0.28, y=0.40.

Polychromatic white light may also be defined according to the ANSI standard C78.377-2008 using the Planckian locus (or black body line) where the white light area for the range of CCTs between 2700K to 6500K is described by the length of the white CCT lines which are each 0.012 Duv (+−0.006 Duv from the Planckian locus).

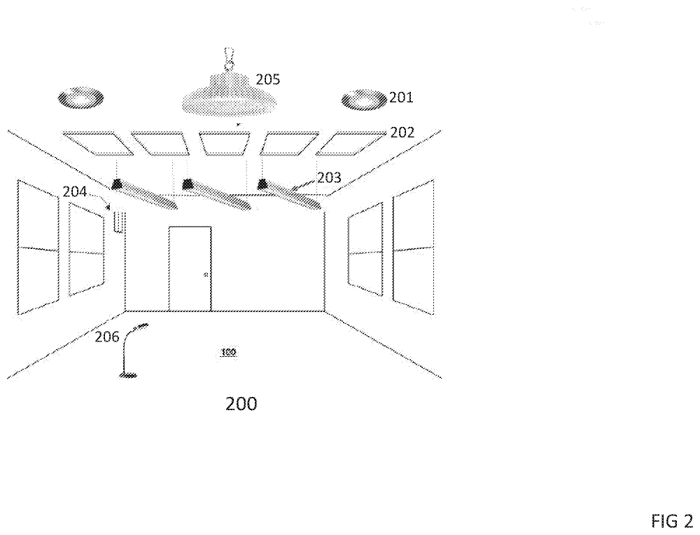

Typical indoor electrically illuminated environments commonly use indoor electrical light sources and light fixtures including recessed troffers, pendant fixtures, downlights, high bays, indirect lights, indirect/direct lights, cove mounted lights, wall sconces, task lamps, desk lamps, and light bulbs as shown in FIG. 2. Outdoor lights used at night include down floods, up floods, bollard lights, street lights, parking lot lights.

The Circadian Potency SSD disclosed here is of importance to the design of lighting that promotes robust circadian rhythm entrainment, and human health, safety, mood and productive performance, while still meeting the color rendering, aesthetic and visual requirements for task and safety illumination.

The visual appearance, color rendering, aesthetic and other requirements for task and safety illumination are defined in lighting standards published by the Illuminating Engineering Society (IES) and other standards organizations including the European standard EN 12464-1. These generally accepted standards generally require that workplaces are illuminated by Polychromatic White Light with a Color Rendering Index (CRI) of greater than 80, and a table top light intensity of 200-1,000 lux depending on work task and/or other activities or applications.

To determine the Circadian Potency of light sources and illuminated environments and their suitability for day and night applications requires the use of an Circadian Potency SSD that is appropriate and applicable for illuminated environments that have Polychromatic White Light with a Color Rendering Index (CRI) of greater than 80, and a table top intensity of 200-1,000 lux depending on work task and other activities.

Unfortunately, there are fundamental flaws in the Relative Circadian SSDs currently used in the field of lighting by lighting manufacturers, lighting designers, academic researchers and others. One, named Circadian Stimulus (CS), is based on short (30-90 minute) exposures of dark adapted eyes to monochromatic lights, and the other, named Equivalent Melanopic Lux (EML) is a theoretical prediction based on the photochemical properties of the retinal melanopsin photopigment corrected for pre-receptoral filtering (CIE TN 003:2015). The CS and EML functions have significantly divergent non-compatible Relative Circadian SSDs. The CS and EML thus provide significantly divergent guidance from each other and from the Circadian Potency SSD disclosed herein.

Importantly, neither the CS nor the EML Relative Circadian SSD has been empirically developed using human subjects illuminated by Polychromatic White Light with the SPDs, the light intensities, or the exposure durations to which people are typically exposed, whereas the Circadian Potency SSD was empirically derived using normal illumination conditions of white light at the intensity and duration to which people are typically exposed. The Circadian Potency SSD disclosed herein is not anticipated or previously known.

When scientific evidence is discovered that is not compatible with or predicted by either the CS or the EML Relative Circadian SSDs it has been generally dismissed by the academic researchers working in the field. Accordingly, the Circadian Potency SSD is non-conventional, and prior approaches teach away from the Circadian Potency SSD.

However, the Circadian Potency SSD is consistent with investigations of circadian effects conducted under conditions approximating real world conditions. For example, Rahman et al (2011) from the University of Toronto conducted research of healthy human subjects spending 12-hour night shifts under typical polychromatic white light illumination at intensities typically seen in the workplace. As will be discussed more fully below, Rahman et al found that when light wavelengths below 500 nm were filtered out, full circadian function was restored. This finding has been subsequently confirmed by Gil-Lozano et al (2017).

The research findings of Rahman et al (2011) and Gil-Lozano et al (2016) would not be predicted by the CS or EML models since both the CS and the EML Relative Circadian SSDs show considerable circadian spectral sensitivity at wavelengths of 500 nm and above. Thus this finding of Rahman et al and Gil-Lozano et al obtained under normal workplace white lighting conditions, is incompatible with the predictions from either the CS and EML Relative Circadian SSD models. However, these research findings are predicted by the Circadian Potency SSD shown herein.

It is noteworthy that neither the CS or EML models were derived using human subjects illuminated by polychromatic white light with the spectra, the intensities, or the durations to which people are typically exposed.

However, the Circadian Potency SSD, in contrast to the EML and CS was derived using data obtained under these normal illumination conditions. In various embodiments, the novel finding of the empirically derived Circadian Potency SSD for human subjects illuminated by polychromatic white light, where data indicating the spectra, intensity, and duration to which people are exposed was tracked. This Circadian Potency SSD is significantly different from the CS or EML Relative Circadian SSDs.

It is noteworthy that the Circadian Potency SSD is compatible with the findings of Rahman et al and Gil-Lozano et al from human subjects illuminated by polychromatic white light with the spectra, intensity, and duration to which people are typically exposed. However, the data of Rahman et al and Gil-Lozano et al does not provide sufficient information to derive the Circadian Potency SSD provided herein.

The newly derived Circadian Potency SSD disclosed herein enables novel lighting sources, lighting systems, lighting methods, control systems, and lighting sensing systems, as well as other related implementations.

To determine and derive the Circadian Potency SSD required us to empirically define the precise wavelength range which must be stimulated by light during the day and must not be stimulated during hours of darkness from sunset to sunrise under the illumination conditions that meet the lighting standards prescribed for workplaces and other human occupied environments.

To measure the relative stimulation of the human circadian timing system by light of different wavelengths required the selection of one or more biomedically relevant assay markers of human circadian function based on the structure and function of the human circadian timing system and the pathophysiology of circadian disruption by light at night.

Specialized retinal photoreceptors activate neural and endocrine visual pathways which mediate the synchronization of the human circadian clock in the suprachiasmatic nucleus (SCN) to environmental day-night schedules, and synchronize the circadian clocks and the circadian rhythms in the pineal gland, pituitary, and in other diverse neural, neuroendocrine, metabolic and carcinogenic processes.

Among the key endocrine regulators used by the SCN to transmit transduced light-dark and circadian phase information to the systems of the body and initiate reparative and other protective functions at night are the neurohormone melatonin and the adrenal hormone cortisol.

Melatonin (N-acetyl-5-methoxytryptamine) is the principal hormone of the pineal gland, and mediates many biological functions, particularly the timing of those physiological functions that are controlled by the duration of light and darkness. Melatonin is synthesized from tryptophan through serotonin, which is N-acetylated by the enzyme n-acetyl transferase or NAT, and then methylated by hydroxyindol-O-methyl transferase.

The enzyme NAT is the rate-limiting enzyme for the synthesis of melatonin, and is increased by norepinephrine at the sympathetic nerve endings in the pineal gland. Norepinephrine is released at night or in the dark phase from these nerve endings. Thus, melatonin secretion may be strongly influenced by the daily pattern of light and dark exposure.

The release of high levels of melatonin during darkness at night is essential to healthy body functions. Melatonin has been shown to have various functions such as chronobiotic regulation, immunomodulation, antioxidant effects, regulation of the timing of seasonal breeding and oncostatic effects (Reiter et al 2016).

Evidence of oncostatic effects of melatonin that have been shown in vitro, and in animal studies, suggest a key role in suppressing tumors and protecting against the proliferation of cancer cells, including human breast and prostate cancer.

Low levels of nocturnal melatonin release may be associated with breast cancer, prostate cancer, type 2 diabetes, metabolic syndrome, insulin resistance, diabetic retinopathy, macular degeneration, hypertension, coronary artery disease, congestive heart failure, depression, anxiety, migraines and other life threatening or debilitating conditions.

In recent years, there has been an increasing recognition that melatonin may confer protection from disease, and lower levels of melatonin have been associated with a wide variety of diseases and chronic conditions. The scope of this relationship may be potentially far-reaching, and may include cancers, cardiovascular disorders such as hypertension and coronary artery disease, metabolic disorders such as insulin resistance and type II diabetes, Huntington's disease, multiple sclerosis, Alzheimer's disease, migraine headaches, and psychiatric disorders such as depression and anxiety, etc. In some diseases, such as cancer, there appears to be an inverse linear relationship between melatonin levels and disease risk, such that lower melatonin levels are associated with a significant increase in disease risk. Furthermore, there is no clear “threshold” for this relationship, suggesting that any loss of endogenous melatonin due to light exposure at night would be associated with relatively increased disease risk.

Disruptions in the timing and synchronization of the biological clocks in the multi-oscillator circadian timing system (“Circadian Disruption”) is a fundamental cause of disease processes, pathophysiology and ill-health. A number of useful bio-markers of Circadian Disruption have been established and are used in the field, including the phase, periodicity and amplitude of melatonin levels in body fluids such as saliva, blood and urine.

For this reason, there is a critical need to minimize circadian disruption due to light at night, and to protect the normal phase, period and amplitude of neuroendocrine circadian rhythms such as that of melatonin. Similarly, there may be a need to enhance circadian stimulation during the day.

Melatonin levels in saliva, blood or urine can also serve as a key marker of circadian clock timing that can be used in studies of the effects of light on the timing of circadian clocks. However, it is important to distinguish between transient changes in melatonin levels caused by short exposures of light at night which may not cause changes in the timing of circadian clocks or any significant health effects, versus sustained effects of light on the timing of the rhythms of melatonin release which indicate phase shifts or other significant changes in the function of circadian clocks.

There are serious limitations in the methods that have been previously used to attempt to determine the Relative Circadian SSD, which have created considerable debate and uncertainty among scientists and lighting industry practitioners of what is the effective wavelength range for optimal circadian effectiveness under the normal lighting conditions for human-occupied built environments.

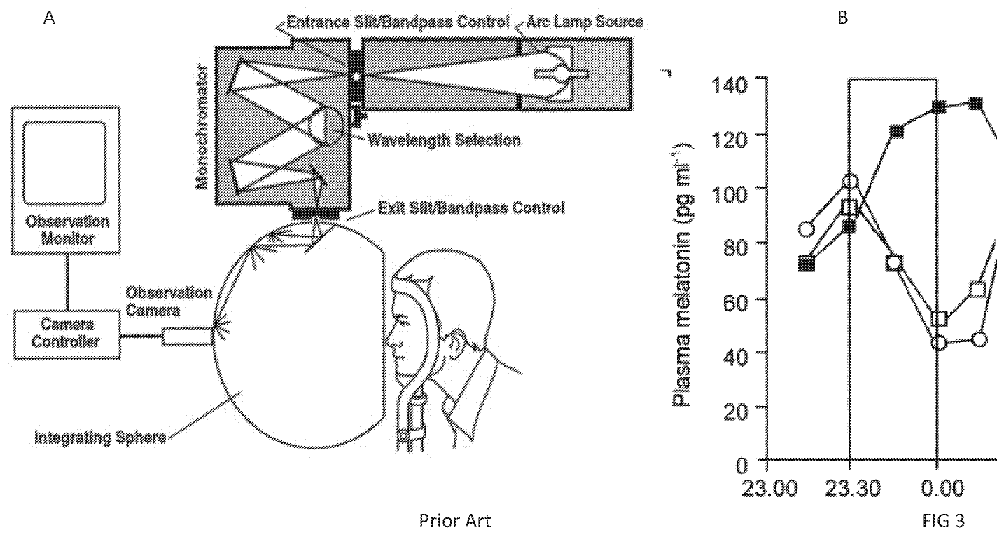

Some investigators have used laboratory experiments where human subjects with pharmacologically-dilated pupils are placed in a fully dark-adapted state by wearing blindfolds for two hours in a darkened laboratory and then are exposed to short (30-90 minute) durations of monochromatic light of different colors. For example, Thapan et al (2001) used 30 minutes of monochromatic light exposure, and Brainard et al (2001) used 90 minutes of monochromatic light to determine short-term effects on melatonin suppression. In these studies, as shown in FIG. 3A, the subjects typically have their heads constrained by a chin stirrup, and are asked to stare into a light box, and therefore cannot move freely or perform normal work tasks. The measurement of melatonin level is made before, during and after the short light exposures. (FIG. 3B). This data derived under these artificial conditions was used to derive Circadian SSD models such as the CS model.

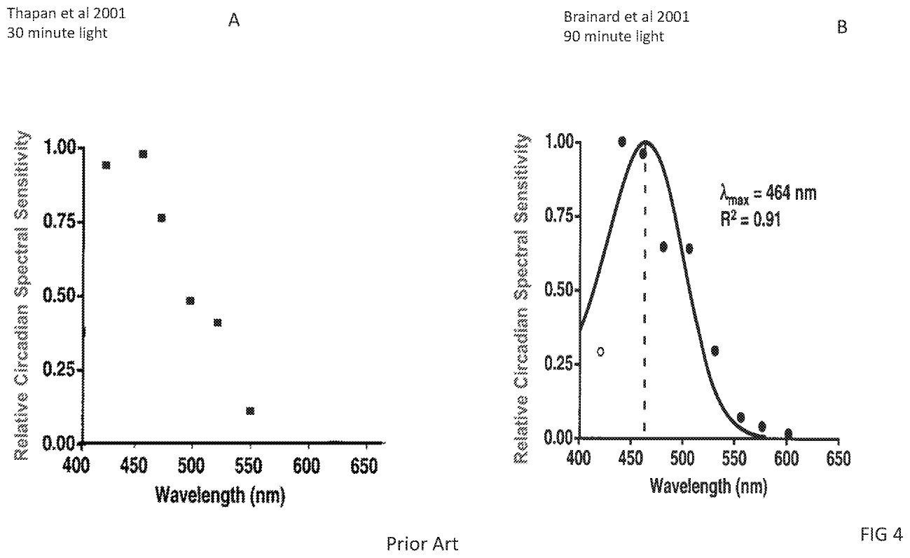

A comparison of the Relative Circadian SSDs in FIG. 4 that were derived from these short monochromatic light exposure studies show that there are significant changes in spectral sensitivity particularly between 400 and 440 nm as the experimental exposure to monochromatic light was increased from 30 minutes to 90 minutes suggesting that there are significant transients in the Relative Circadian SSDs. For example, at the shortest wavelengths of monochromatic light tested (420-424 nm), Thapan (FIG. 4A) found 95% of maximum melatonin suppression with 30 minutes of light duration while Brainard (FIG. 4B) found that the suppression was only 27% of maximum suppression with 90 minutes of light exposure.

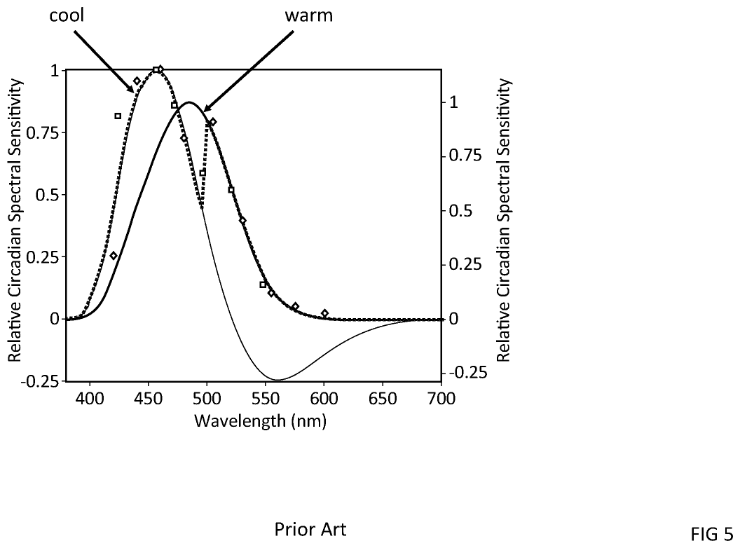

Relative Circadian SSDs have been derived from this short-term light exposure and short-term melatonin responses such as the Circadian Stimulus (CS) functions proposed by Rea et al (2005). The Relative Circadian SSDs of CS proposed by Rea for warm (Coordinated Color Temperature (CCT) less than approximately 3300 K), and cool CCT (CCT greater than approximately 3400 K) are shown in FIG. 5.

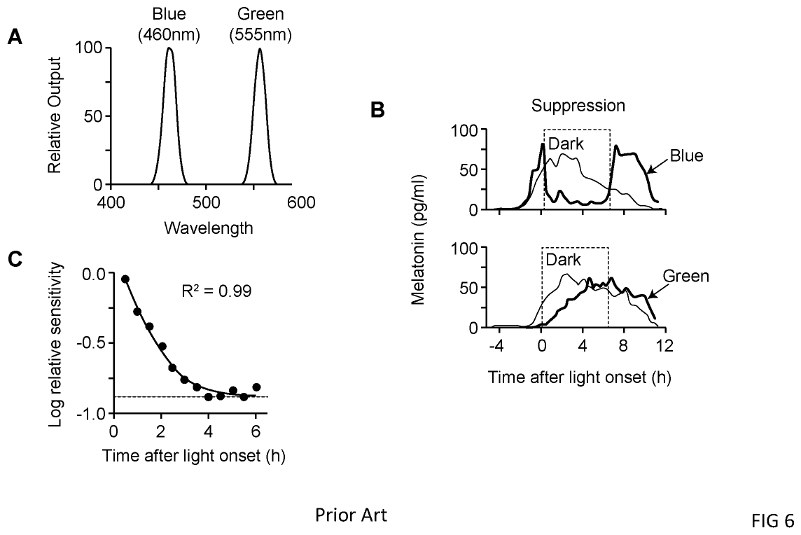

Distinguishing the transient effects of short exposures of light from sustained or circadian effects is important for the design of spectrally-engineered lights. For example, Gooley et al. showed that in a protocol with an extended 6-hour exposure to either monochromatic green (555 nm) or blue (460 nm) light (FIG. 6A), that both the green and blue light had a similar suppressive effect on melatonin over the first hour (FIG. 6B). The green-light-induced melatonin suppression rapidly decayed while the blue light exposure caused a sustained suppression of melatonin. The decay in the effect of green light on melatonin suppression was completed over the first 2-3 hours of light exposure from a dark-adapted state as is shown in FIG. 6C.

These transient effects of light on human physiology which appear to be largely mediated by the cones in the retina (Lucas et al 2014) may not be relevant to the maintenance of circadian period and/or phase, the optimal synchronization of the human circadian timing system, or the prevention of circadian disruption and the associated adverse effects on human health.

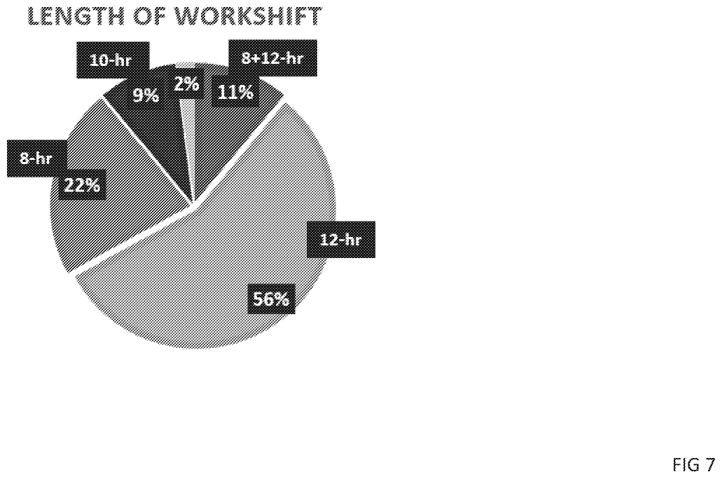

The experimental conditions of the short monochromatic light treatment studies are very different from, and do not represent, the normal conditions of illumination where people in indoor built environments move around freely under polychromatic white light of light intensities between 200 and 1000 lux at table top during work shifts of 8 to 12 hours in length. As shown in FIG. 7, a recent survey of 224 employers showed that 56% of employees work 12-hour shifts, 9% work 10-hour shifts, 22% work 8-hour shifts, and 11% work a combination of 8-hour and 12-hour shifts.

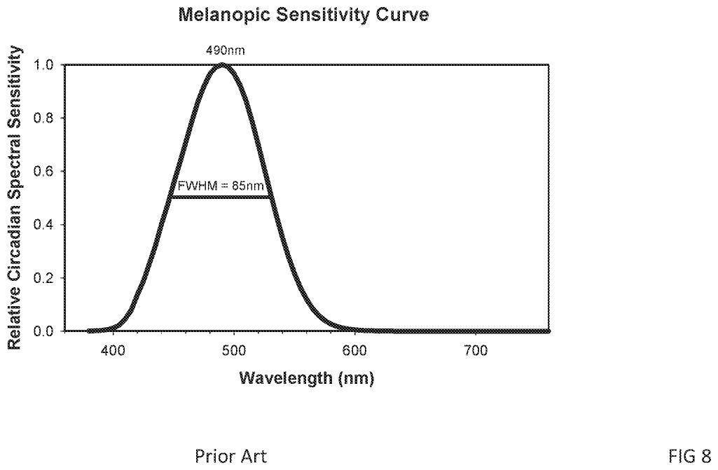

Other investigators have used the spectral sensitivity characteristics of a known photoreceptor pigment such as melanopsin determined in in vitro experiments in tissue cultures to attempt to define an alternative Relative Circadian SSD of the human circadian system. An example of a Relative Circadian SSD derived from this data is the Equivalent Melanopic Lux (EML) model proposed by Lucas et al (2014). The spectral sensitivity function predicted by EML uses the spectral sensitivity of melanopsin and applies a correction for pre-receptoral filtering (CIE TN 003:2015) to generate a Relative Circadian SSD with a peak sensitivity at 490 nm and a symmetric FWHM of 85 nm as shown in FIG. 8.

The limitation of the EML is that it relies on the predicted photochemical behavior of a photopigment. Because of the complexity of the circadian photoreception system, the photochemical properties of melanopsin do not necessarily reflect normal overall human circadian spectral sensitivity to light under polychromatic white light. The theoretical spectral sensitivity prediction of EML was not empirically derived under the normal conditions of illumination where people in indoor built environments move around freely under polychromatic white light of light intensities between 200 and 1000 lux at table top during work shifts of 8 to 12 hours in length. The EML model is limited by only a partial understanding of the complex pathways and multiple interacting mechanisms of human physiological systems in real-world environments.

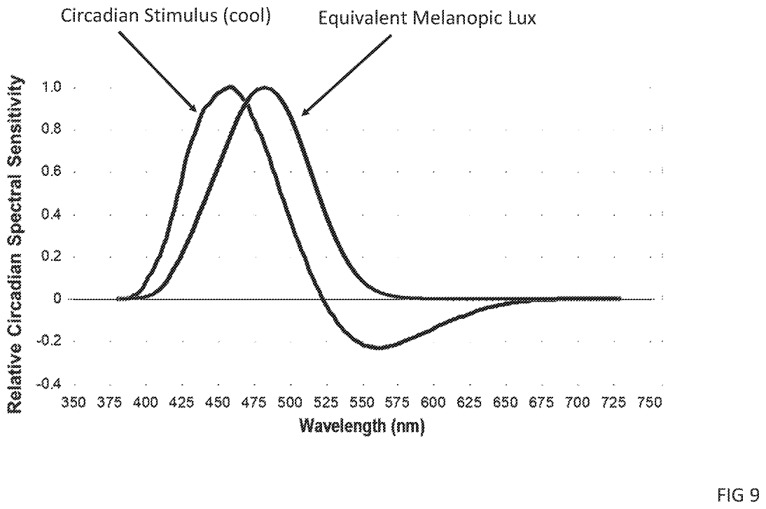

The Relative Circadian SSDs of the two leading models (Circadian Stimulus (CS) and Equivalent Melanopic Lux (EML) that are currently used by lighting manufacturers to design light products and lighting specifiers to select products are significantly different from each other as is shown in FIG. 9. As a result the designers, manufacturers and specifiers of circadian lighting products have become confused by the large difference between the published Relative Circadian SSDs of the CS and EML and other models of circadian sensitivity to light.

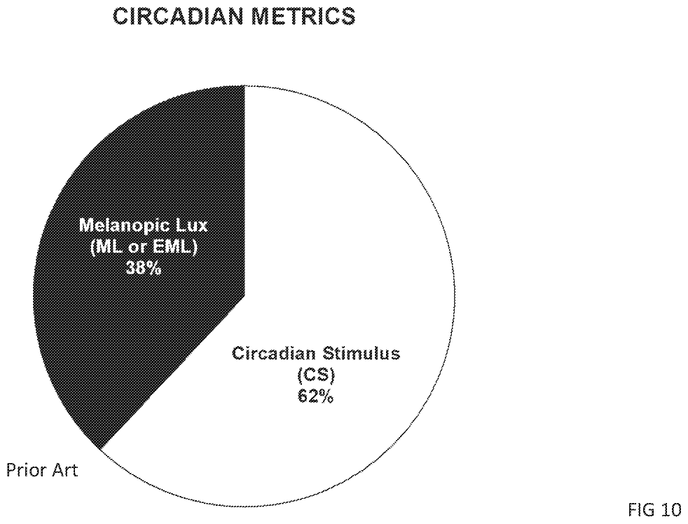

A recent survey of lighting specifiers in the USA showed that 38% used EML and 62% used CS (FIG. 10), and that many lighting designers were confused (Clark and Lesniak (2017). Neither of these Relative Circadian SSD models, nor any other published model, represents the normal conditions of illumination to which people in indoor environments are exposed, where they move around freely under polychromatic white light of light intensities between 200 and 1000 lux during work shifts of 8 to 12 hours in length.

Current lighting products which are intended to improve human alertness, performance, mood and health by maximizing the stimulation of the human circadian system during the daytime and/or minimizing circadian stimulation at night are thus based on Relative Circadian SSDs derived in experimental conditions which are not representative of normal human exposure to environmental and electric light, and furthermore are based on untested assumptions of human circadian function.

Disclosed herein is the experimentally derived and previously unreported Circadian Potency SSD of the human circadian system when people are exposed to polychromatic white light under normal working and living conditions.

Unlike previously reported attempts to define the spectral sensitivity of the human circadian system, experimental protocols conducted under conditions representative of normal lighting conditions were used to obtain the data to derive the Circadian Potency SSD disclosed herein, Applicants used a) polychromatic white light instead of the monochromatic light stimuli, previously used in other studies; b) normal work shift length light exposures of 12 hours in duration instead of brief 30-90 minutes light exposure previously used in other studies, c) a representative level of illumination recommended for workplaces by IES of 540 lux (50 foot candles) table top instead of the light levels used in other studies that did not represent light levels normally used in workplaces; d) measurement of the total 12-hour night suppression of melatonin (area under the melatonin curve) as a physiological indicator of circadian stimulation that has direct relevance to health outcomes instead of measurements of brief transient suppressions of melatonin in previous studies that may not be related to health outcomes.

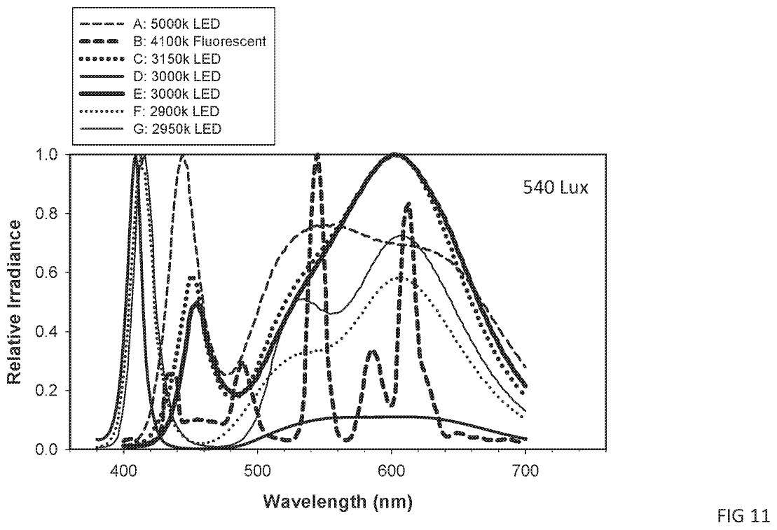

To obtain this novel and accurately predictive human Circadian Potency SSD function human subjects were studied during a series of simulated 12-hour night shifts while illuminated at 540 lux table top intensity levels by overhead ceiling illumination from polychromatic white sources selected from a set of fluorescent and LED light sources with diverse spectral power distributions and CCT values ranging between 2700K and 5000K. The spectral power distributions of various white color sources used in these tests are shown in FIG. 11.

In an experiment, salivary melatonin samples for each human subject were collected at hourly intervals between 8 pm and 8 am for each night of exposure under each of the selected white light sources and analysed for melatonin concentration using standard laboratory procedures. The area under the curve (AUC) of the curve of melatonin levels during the nocturnal rise and fall of melatonin concentration between 8 pm and 8 am was calculated for each individual subject, and the mean melatonin AUC for groups comprising 7 to 12 individuals for each lighting condition was determined.

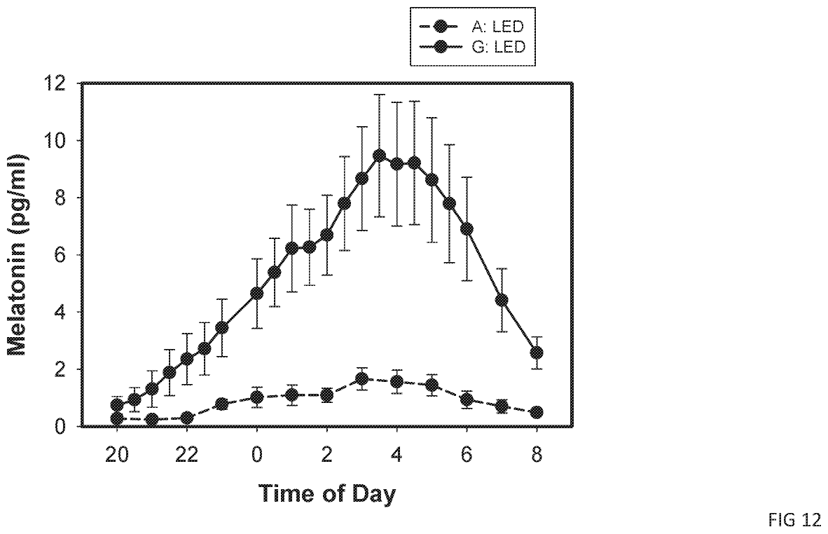

FIG. 12 shows examples of two of the mean melatonin area under the curve (AUC) functions comparing a white SPD light with low melatonin suppressive effect (Source G) and a white SPD light with high melatonin suppressive effect (Source A).

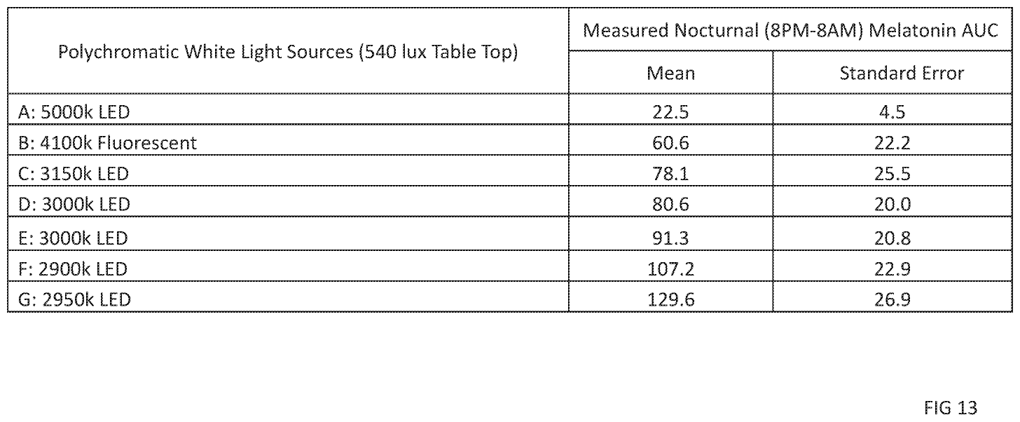

The results reported in FIG. 13 are based on the initial analysis of a total of 67 human subject nights of data. There was a substantial range of over five-fold variation in the mean nocturnal melatonin AUC with different light SPDs at the same light intensity. The melatonin AUC ranged between 22.5 and 129.6 h*pg/ml depending on the Relative SPD380-780 of the selected white light source each of which was maintained at the same light intensity of 540 lux table top for 12 hours.

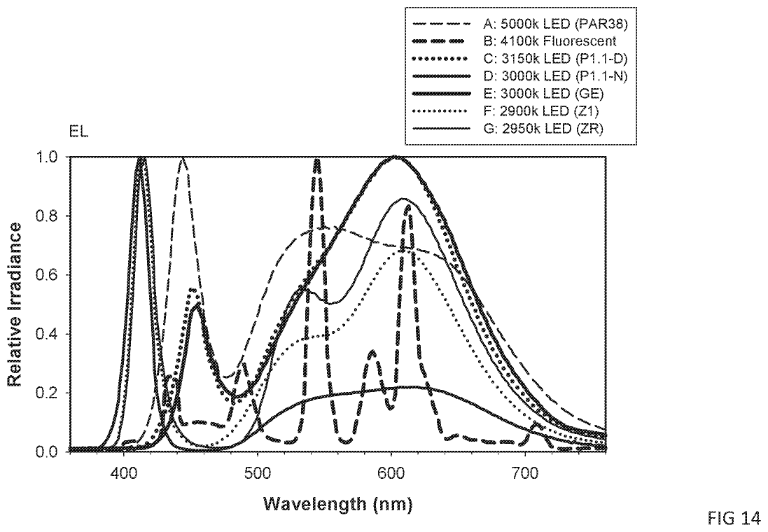

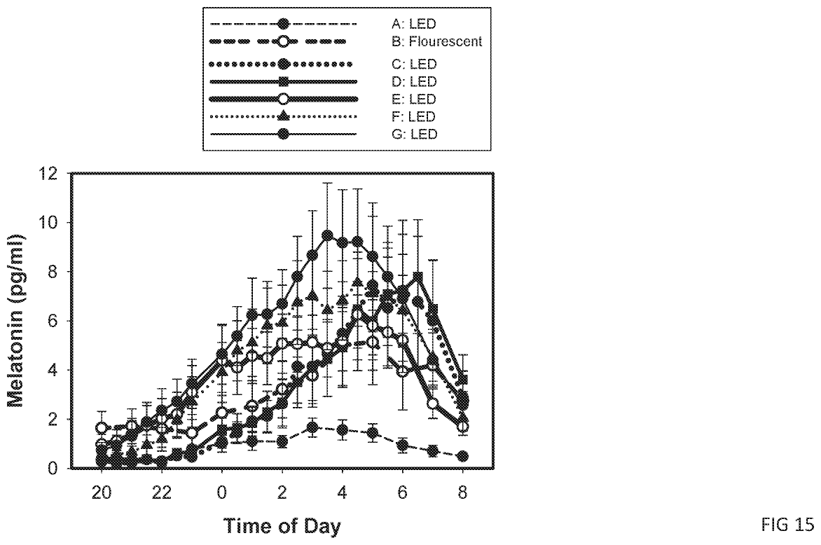

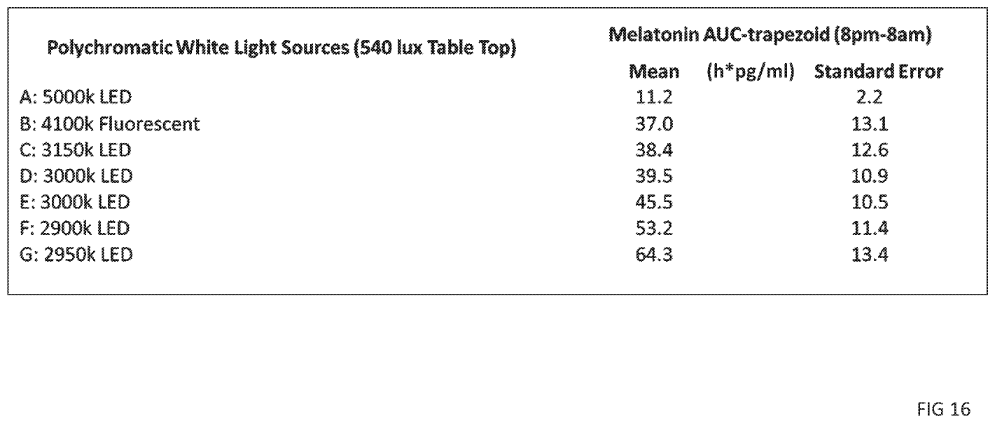

A subsequent more comprehensive analysis of the experimental data is disclosed in this application where the vertical illuminance was measured at eye level facing forwards to determine the corneal eye-level SPD under each lighting condition (FIG. 14), and the melatonin AUC was determined by the trapezoid method for a larger sample of 73 human subject (47 male/26 female aged 20-41) nights of exposure (FIG. 15). In this analysis we found a greater than five-fold variation in the mean nocturnal melatonin AUC between the different light SPDs at the same light intensity. The melatonin AUC ranged between 11.2 and 64.3 h*pg/ml depending on the relative SPD of the selected white light source (FIG. 16) even though each were maintained at the same light intensity of 540 lux table top for 12 hours which provided 250-270 lux vertical illuminance at the cornea of the eye.

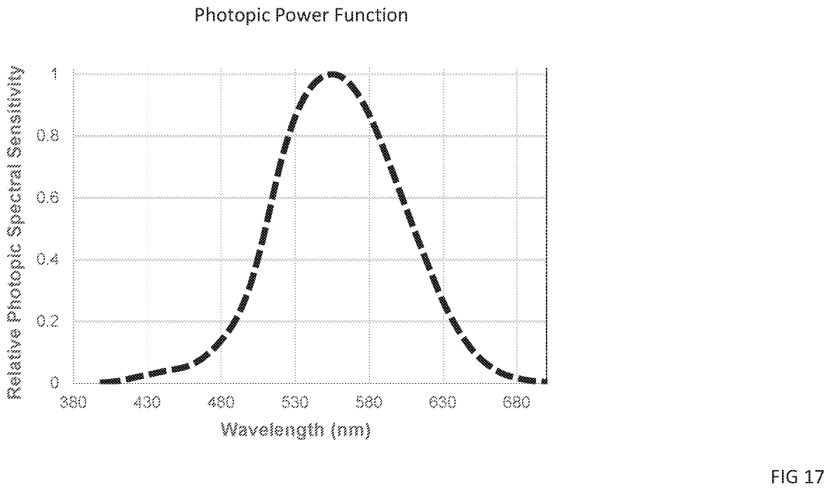

The traditional metrics of lux and lumens are based on the visual properties of the visual cones in the retina using a photopic function which has a peak at 555 nm and a FWHM of 100 nm between 510 and 610 nm. (FIG. 17). This photopic curve is widely used in the lighting industry (for example in light meters) to assess the brightness or intensity of light. However, it is generally accepted that it does not describe the relative spectral sensitivity distribution of the human circadian system to light.

For each light source studied the normalized spectral power distribution between 380 and 780 nm was multiplied by the photopic power at each wavelength to calculate the relative total photopic power between 380-780 nm (Relative Total Photopic Power380-780) of each light source.

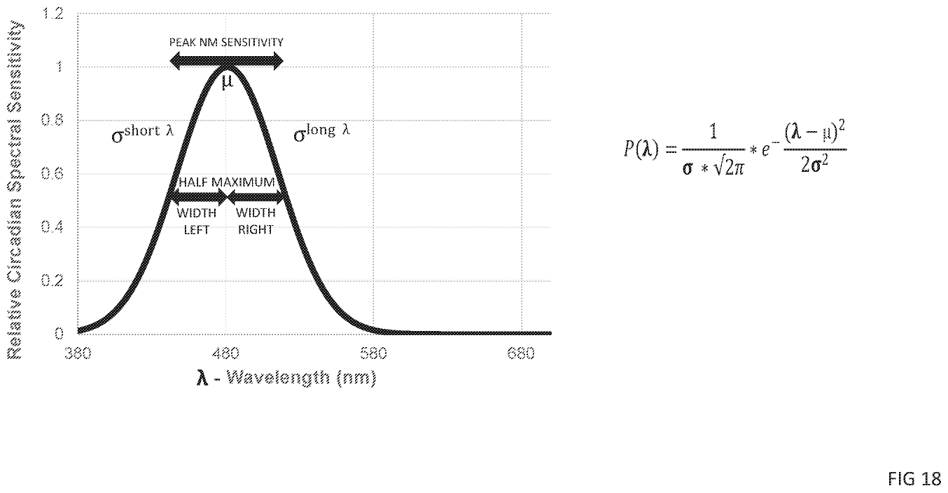

The Circadian Potency SSD function was derived by correlating the AUC melatonin values against the Relative SPD380-780 of the various light sources using an optimization routine. Three independent parameters: (1) peak Circadian Potency SSD wavelength, (2) optimized Gaussian curve fit to wavelengths lower than the peak Circadian Potency SSD wavelength and (3), and optimized Gaussian curve fit to wavelengths higher than the peak Circadian Potency SSD wavelength, were used in the Circadian Potency SSD function optimization routine. FIG. 18 shows an exemplar curve for circadian spectral sensitivity and the three independent optimization parameters which allowed for a wide variety of possible curve shapes and possible wavelengths of the maximum relative circadian sensitivity wavelength to be investigated.

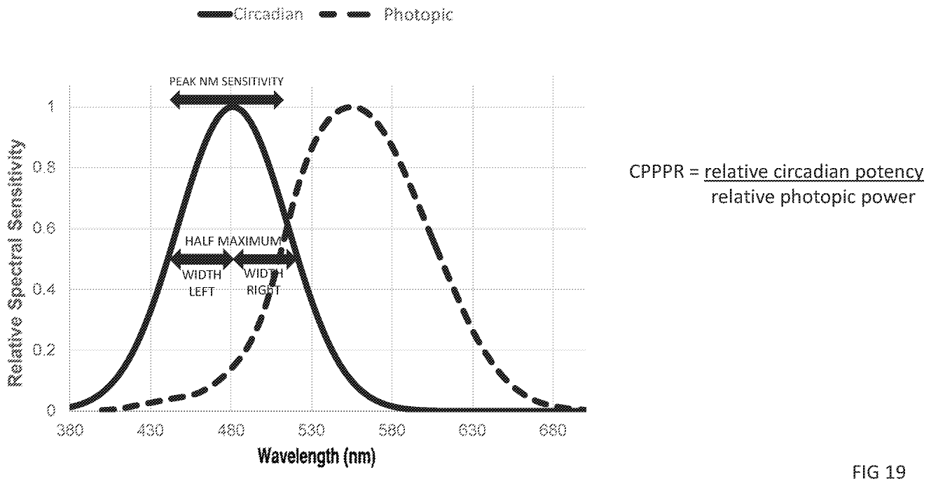

The optimization routine calculated a total Circadian Potency SSD function, and then determined the ratio between the total relative Circadian Potency SSD power and the Relative Total Photopic Power 380-780 (“Circadian Potency/Phototopic Power Ratio (CPPPR)”) for multiple iterations of the three parameters which described each potential Circadian Potency SSD best fit curve as shown in FIG. 19.

These CPPPR values for each potential Circadian Potency SSD curve fit was iteratively correlated against the melatonin AUC results across the set of light sources until a best fit regression function between CPPPR and melatonin AUC was found.

Optimal curve fits to the data were calculated using the Gaussian function:

$$P ( λ ) = \frac{1}{σ * √ 2 π} * {e}^{- \frac{{( λ - μ )}^{2}}{2 {σ}^{2}}}$$

where
P=Relative Circadian Potency
λ=Wavelength
μ=Peak Circadian Potency wavelength
σ=standard deviation
σ²=variance,

The final asymmetric curve is assembled by:

Choosing the value from the “short wavelength” Gaussian curve if λ<=μ

$$P ( λ ) = \frac{1}{33.5 * √ 2 π} * {e}^{- \frac{{( λ - 478.3 )}^{2}}{2 * {33.5}^{2}}} .$$

Choosing the value from the “long wavelength” Gaussian curve if λ>μ

$$P ( λ ) = \frac{1}{8.9 * \sqrt{2 π}} * {e}^{- \frac{{( λ - 478.3 )}^{2}}{2 * {8.9}^{2}}} .$$

In the subsequent more comprehensive analysis of the experimental data, disclosed in this application, where the vertical illuminance was measured at eye level facing forwards to determine the corneal illuminance and SPD under each lighting condition, and the melatonin AUC was determined by the trapezoid method for a total of 73 subject nights, the short wavelength function was:

$$P ( λ ) = \frac{1}{31.7 * √ 2 π} * {e}^{- \frac{{( λ - 472.2 )}^{2}}{2 * {31.7}^{2}}}$$

In this subsequent more comprehensive analysis the long wavelength function was:

$$P ( λ ) = \frac{1}{9.2 * \sqrt{2 π}} * {e}^{- \frac{{( λ - 472.2 )}^{2}}{2 * {9.2}^{2}}}$$

Before assembling the optimized best fit Circadian Potency curve both sides were scaled so that the maximum Relative Circadian Potency P μ is equal to 1.0. In an embodiment, the circadian day optimized peak is at about 472 nm, and 76% of spectral power is emitted within a full width half maximum (FWHM) range of the circadian day optimized peak between about 435 nm to about 483 nm. In an embodiment, the left side of the negatively skewed asymmetric distribution has a standard deviation (a) value of 30, 31, 32, 33, 34, or 35. As noted above, the curve can be a negatively skewed asymmetric distribution that is a mirror image log-normal distribution, or can include a first half-Gaussian function and a second half-Gaussian function.

Additional variations are possible, for example, where the circadian day optimized distribution includes additional lighting components to bring the lighting to overall white or near-white, for example, where the spectral power distribution further includes greater than approximately 20% of total 380-780 nm visible irradiance in the 440-490 nm wavelength band.

In an embodiment, the circadian day optimized peak is selected from the group of wavelengths consisting of 467, 468, 469, 470, 471, 472, 473, 474, 475, 476, 477 nm. The peak may vary.

In an embodiment, the circadian optimized peak is selected from the group of wavelengths consisting of 480, 481, 482, 483, 484, 485, 486, 487, 488, and 489 nm when illuminated environment occupants include at least one occupant whose age is greater than 50 years of age.

In an embodiment, the light source includes a light emitting diode comprising a die with a peak emission at 478 nm which is coated with phosphors adapted to provide a strong photopic emission between 500 nm and 700 nm.

In an embodiment, the polychromatic white or near white light is provided within an oval space on the CIE 1931 chromaticity diagram with the coordinates of the long axis between x=0.47, y=0.45 and x=0.21, y=0.26, and coordinates of the short axis between x=0.31, y=0.40 and x=0.37, y=0.30. Applicant notes that the white area includes shoulder regions which may not be captured in the oval space, and in some embodiments, the polychromatic white or near white light is provided within any space, including the shoulders beyond the oval, on the CIE 1931 chromaticity diagram that are white or near-white.

In an embodiment, the light source includes an array of red, green and blue light emitting diode chips where the blue light emitting diode has a peak emission at approximately 475-480 nm, which, in combination, are powered to create the circadian-optimized polychromatic light that is substantially white.

In another embodiment, the polychromatic white or near white light is provided defined according to the ANSI standard C78.377-2008 using the Planckian locus (or black body line) where the white light area for the range of CCTs between 2700K to 6500K is described by the length of the white CCT lines which are each 0.012 Duv in length (and are +−0.006 Duv from the Planckian locus). As mentioned earlier, a white or near-white light may be desirable in workplaces where individuals may be used to white light or need contrast and illuminance from white light to perform their work tasks.

In an embodiment, the negatively skewed asymmetric distribution includes at least 78% of an area under a curve defined by the spectral power distribution in the visual wavelength range between about 440 and about 490 nm.

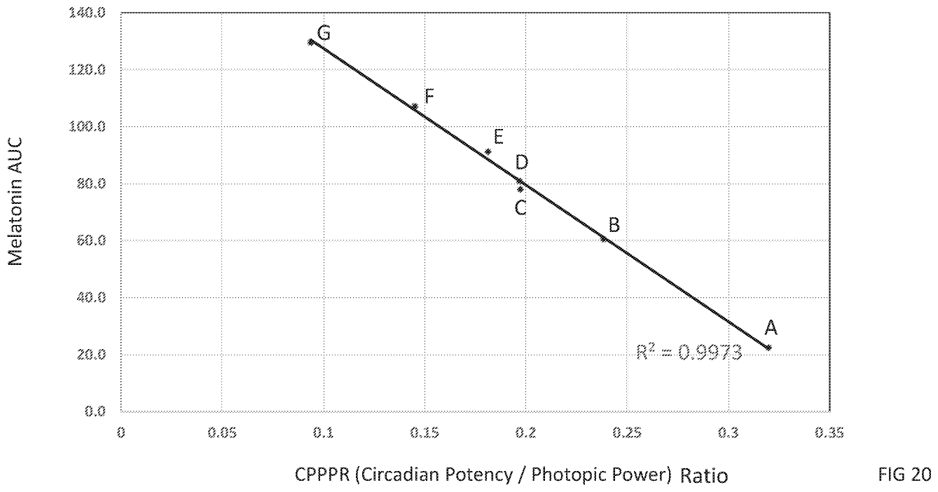

The optimized best fit linear regression function between the CPPPR values of each light source (A-G) and the melatonin AUC for each light source based on an initial analysis of the data is shown in FIG. 20. The light sources had best fit CPPPR values that ranged between 0.09 to 0.33 and these correlated with melatonin AUC values that ranged between 22.5 and 129.6 h*pg/ml. The optimized best fit linear regression of melatonin AUC versus CPPPR which is disclosed in FIG. 20 derived form this initial analysis had a regression R² coefficient of 0.9973 indicating a robust lawful relationship existed in this data collected from 67 human subject nights of white polychromatic light exposure of 540 lux for 12 hours. The high R2 coefficient of 0.9973 for this biological parameter indicates that the Circadian Potency SSD that was derived in these human studies is a precisely described function.

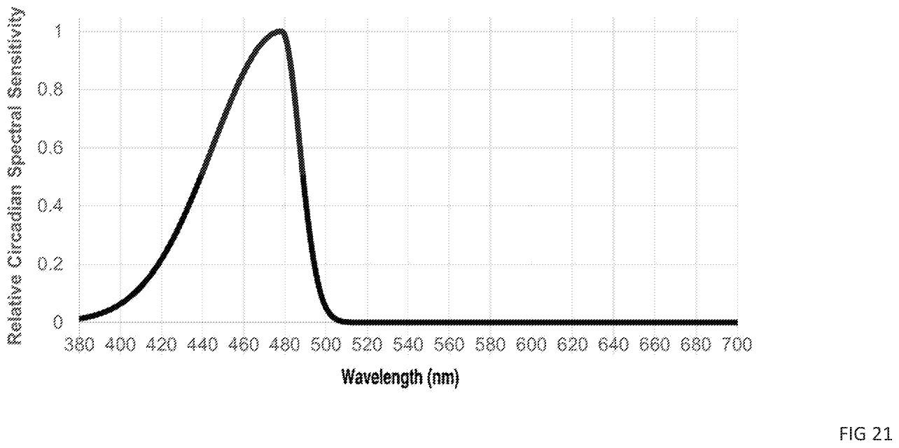

FIG. 21 discloses the best fit optimized Circadian Potency SSD function which provided the best fit melatonin AUC vs CPPPR linear regression line derived from the initial analysis shown in FIG. 20. The Circadian Potency SSD was determined to have a peak wavelength of 478.3 nm, right width half maximum of 12 nm, and left width half maximum of 38 nm. Thus, the Circadian Potency SSD had an unexpected and unpredicted FWHM 50 nm range from 440 nm to 490 nm that was asymmetric relative to the 478 nm peak wavelength of Circadian Potency SSD sensitivity.

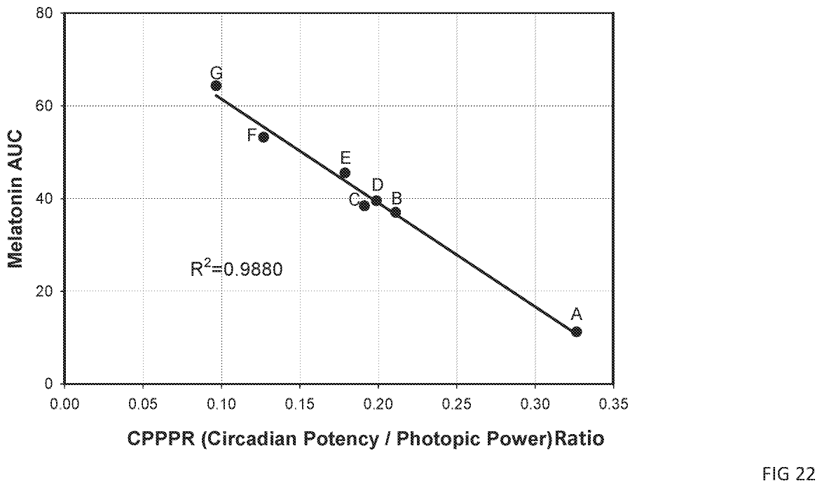

The optimized best fit linear regression function between the CPPPR values of each light source (A-G) and the melatonin AUC for each light source based on the subsequent more comprehensive analysis of the data is shown in FIG. 22 The light sources had best fit CPPPR values that ranged between 0.10 to 0.33 and these correlated with melatonin AUC trapezoid values that ranged between 11.2 and 64.3 h*pg/ml. The optimized best fit linear regression of melatonin AUC versus CPPPR which is disclosed in FIG. 22 derived from this subsequent more comprehensive analysis had a regression R² coefficient of 0.988 indicating a robust lawful relationship existed in this data collected from 73 human subject nights of white polychromatic light exposure of 540 lux for 12 hours. The high R² coefficient of 0.988 for this biological parameter indicates that the Circadian Potency SSD that was derived from the subsequent more comprehensive of the data from these human studies is a precisely described function.

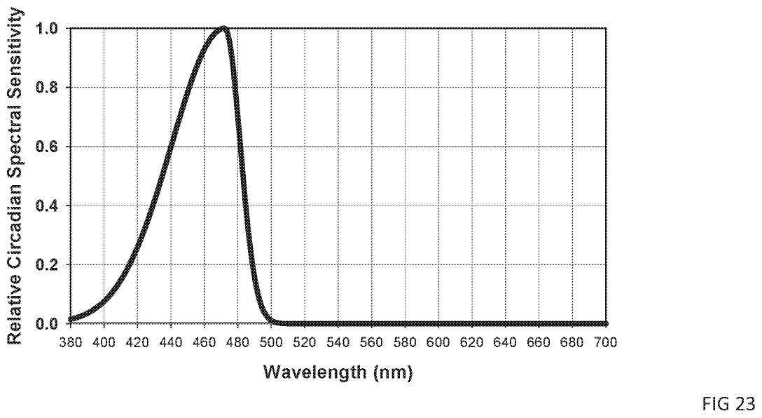

FIG. 23 discloses the best fit optimized Circadian Potency SSD function which provided the best fit melatonin AUC vs CPPPR linear regression line derived from the subsequent more comprehensive analysis shown in FIG. 22. The Circadian Potency SSD was determined to have a peak wavelength of 472 nm, right width half maximum of 11 nm, and left width half maximum of 37 nm. Thus, the Circadian Potency SSD had an unexpected and unpredicted FWHM 48 nm range from 435 nm to 483 nm that was asymmetric relative to the 472 nm peak wavelength of Circadian Potency SSD sensitivity.

A wide range of other functions could potentially be used to define the Circadian Potency SSD including polynomial and other functions. However, because regression R² coefficients of 0.997 and 0.988 respectively indicate a robust lawful relationship was achieved using the two Gaussian functions for short and long wavelengths and the peak sensitivity wavelength, this provides a reasonable and useful fit to the data and a sufficiently accurate curve shape to describe the optimum Circadian Potency SSD with high correlative and predictive power.

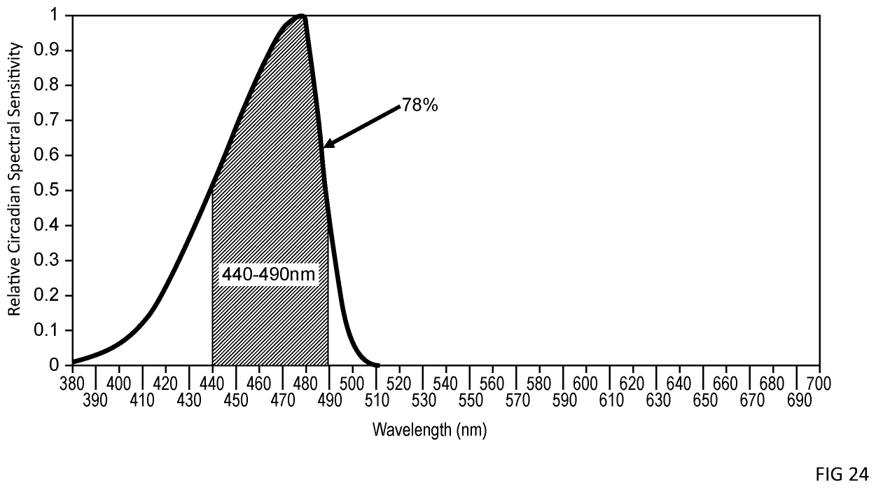

FIG. 24 shows that in the initial analysis 78% of the total Circadian Potency SSD irradiant power was in the 440-490 nm FWHM band. This Circadian Potency SSD from data obtained during extended (12 hour) exposures to polychromatic white light is novel, and not predicted by the results from short term (30-90 minute) exposures to monochromatic light sources, or by the currently used CS or EML models of circadian light effectiveness. It enables the design of unique light sources, filters, light sensors and controls as discussed in more detail below.

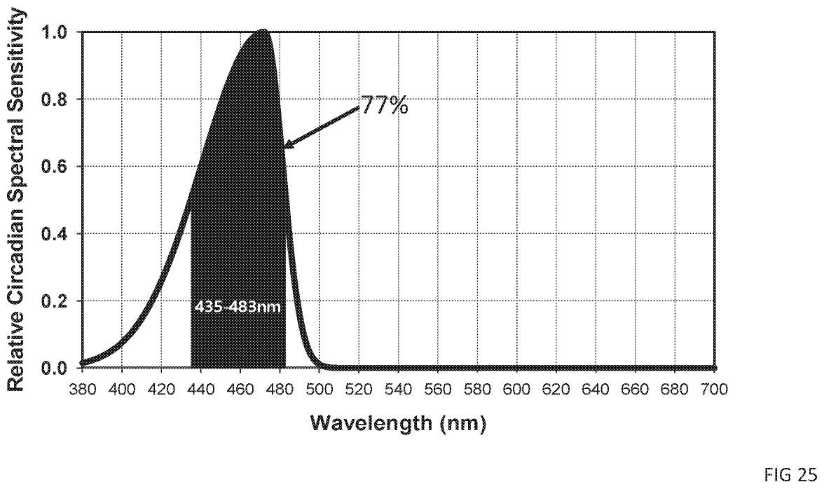

FIG. 25 shows that in the subsequent more comprehensive analysis 76% of the total Circadian Potency SSD irradiant power was in the 440-490 nm band, and 77% was in the 435-483 FWHM band. This Circadian Potency SSD from data obtained during extended (12 hour) exposures to polychromatic white light is novel, and not predicted by the results from short term (30-90 minute) exposures to monochromatic light sources, or by the currently used CS or EML models of circadian light effectiveness. It enables the design of unique light sources, filters, light sensors and controls as discussed in more detail below.

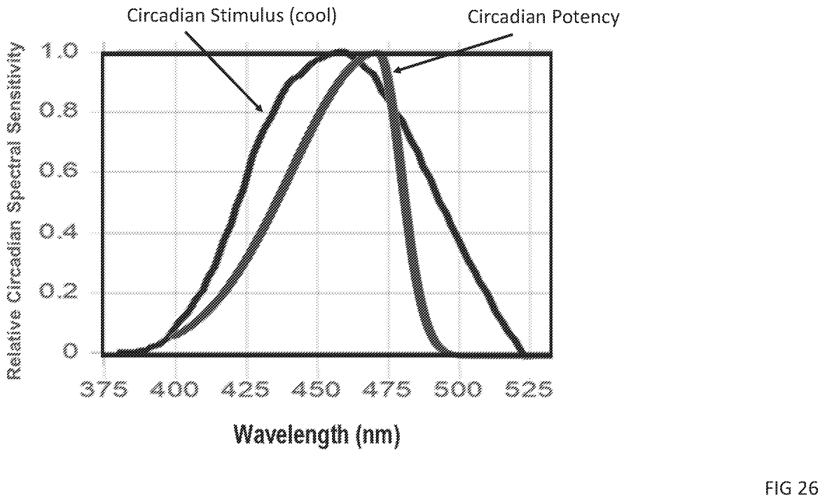

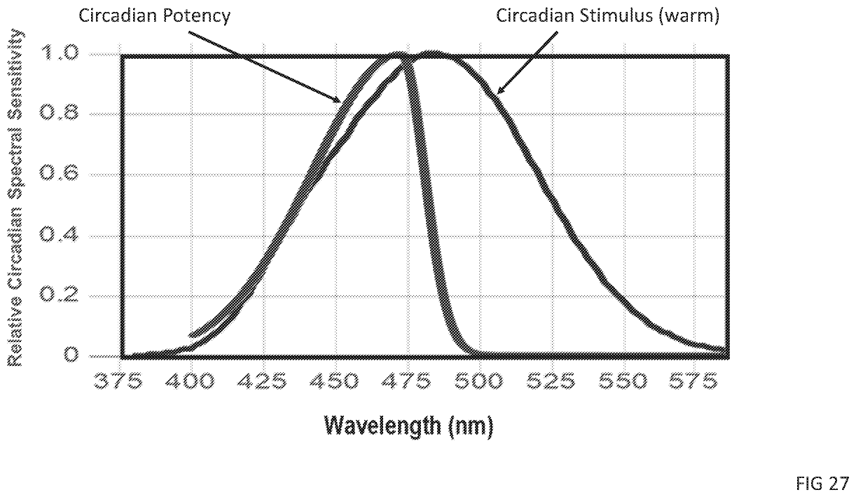

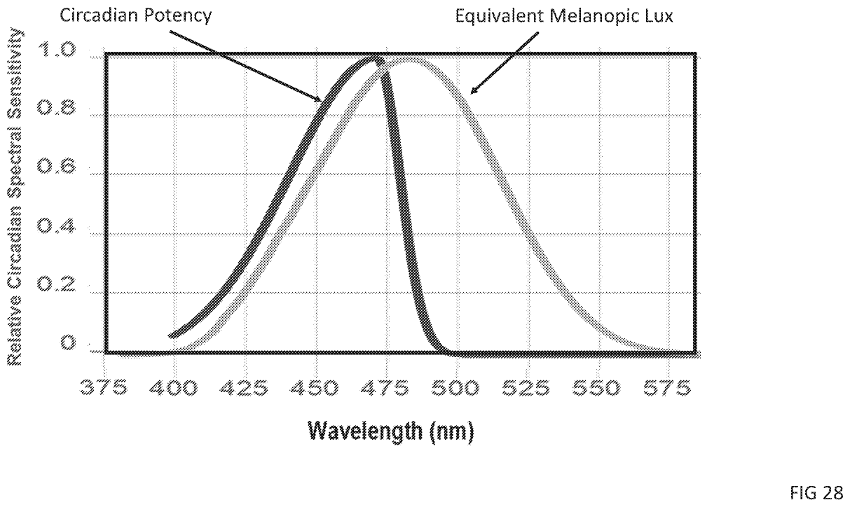

In a comparison of the Circadian Potency SSD with other relative circadian SSD models, FIG. 26 shows that the Circadian Potency SSD disclosed herein is distinctly different from the Relative Circadian SSD used in the Circadian Stimulus model for CCT greater than 3300K CCT (CS cool) of Figueiro and Rea (2017). FIG. 27 shows that the Circadian Potency SSD disclosed herein is distinctly different from the Relative Circadian SSD used in the Circadian Stimulus model for CCT below approximately 3200K CCT (CS warm) of Figueiro and Rea (2017). FIG. 28 shows that the Circadian Potency SSD disclosed herein is distinctly different from the Relative Circadian SSD used in the Equivalent Melanopic Lux model (EML) of Lucas et al (2014).

Previous curve fitting to Relative Circadian SSD data used standard Gaussian functions which assume a symmetric bell curve is the best descriptor of the data set. However, there is no reason to assume such a simple function describes biological reality, and it can result in poor and inaccurately descriptive curve fits.

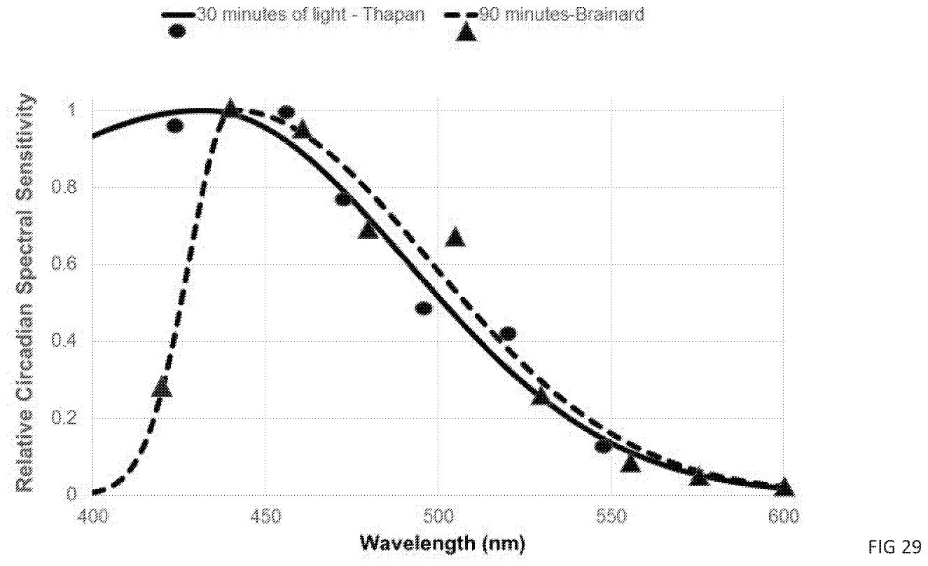

To compare the data from short light exposure and longer light exposure and explore the behavior of transients in the spectral sensitivity response Applicants used a three-parameter optimized curve fitting methodology on all data sets. The experimentally derived data for 30 minute light exposures (Thapan et al 2001) and for 90 minute light exposures (Brainard et al 2001) was analyzed using the same curve fitting optimization methodology where three independent parameters: (1) peak Relative Circadian SSD wavelength, (2) optimized Gaussian curve fit to wavelengths lower than the peak Relative Circadian SSD wavelength and (3), and optimized Gaussian curve fit to wavelengths higher than the peak Relative Circadian SSD wavelength are each optimized to determine the best fit function. This method is superior because it avoids making the incorrect assumption that all Relative Circadian SSDs have a symmetrical Gaussian shape, and allowed for a wide variety of possible best fit curve shapes to be investigated. As shown in FIG. 29, using this three-parameter curve fitting methodology the best fit curve to the 30 minute exposure data of Thapan et al (2001) had a peak spectral sensitivity at 430 nm, and the best fit curve to the 90 minute exposure data of Brainard et al (2001) had a peak spectral sensitivity at 440 nm.

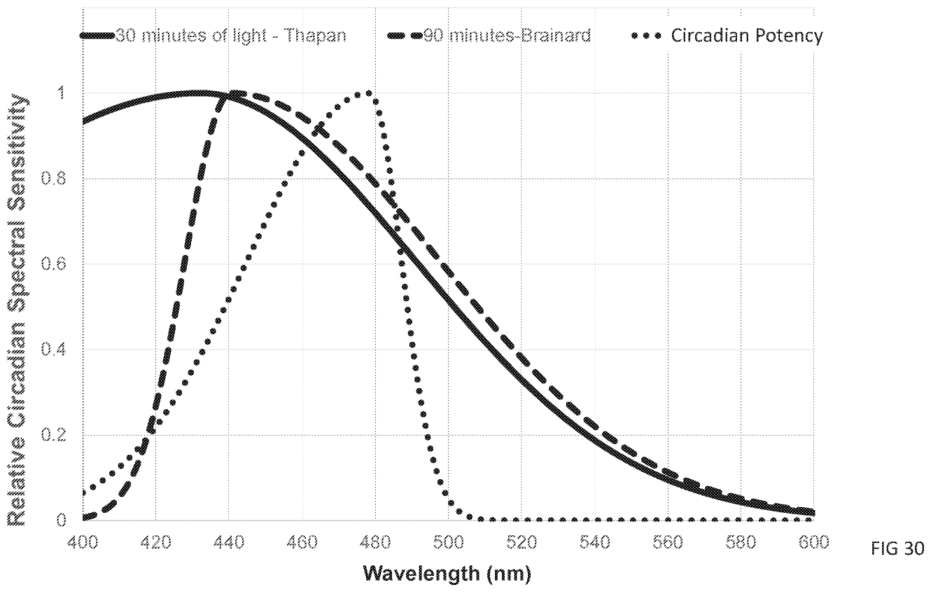

FIG. 30 compares the best fitted Relative Circadian SSDs for 30 minute and 90 minute light exposures with the Circadian Potency SSD function for 12 hours light exposure determined in the initial analysis. It is apparent that there is a progressive transition of the circadian spectral sensitivity distribution function as light exposure is extended from 30 minutes to 90 minutes to 720 minutes (12 hours). The decay of the transient responses to light, which Gooley suggested occurs within the first 2-3 hours of exposure, is associated with a progressive right shift of the maximum circadian sensitivity from 430 nm to 440 nm to 478 nm, and a progressive narrowing of the circadian sensitivity curve from a FWHM of over 100 nm at 30 minutes to 85 nm at 90 minutes to 50 nm once the initial transients have decayed, as exemplified by the 12-hour exposure data of human subject studies. This transition in circadian spectral sensitivity with duration of light exposure is not predicted from previous short monochromatic light treatment data.

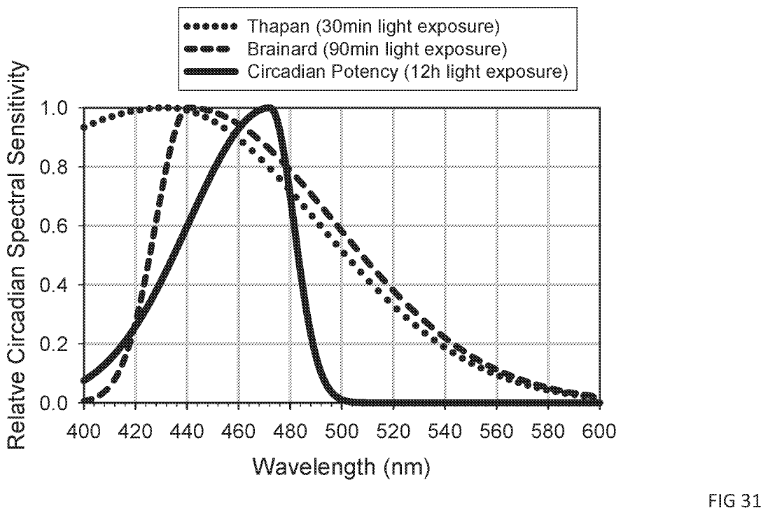

FIG. 31 compares the best fitted Relative Circadian SSDs for 30 minute and 90 minute light exposures with the Circadian Potency SSD function for 12 hours light exposure determined in the more comprehensive subsequent analysis. It is apparent that there is a progressive transition of the circadian spectral sensitivity distribution function as light exposure is extended from 30 minutes to 90 minutes to 720 minutes (12 hours). The decay of the transient responses to light, which Gooley suggested occurs within the first 2-3 hours of exposure, is associated with a progressive right shift of the maximum circadian sensitivity from 430 nm to 440 nm to 472 nm, and a progressive narrowing of the circadian sensitivity curve from a FWHM of over 100 nm at 30 minutes to 85 nm at 90 minutes to 48 nm once the initial transients have decayed, as exemplified by the 12-hour exposure data of human subject studies. This transition in circadian spectral sensitivity with duration of light exposure is not predicted from previous short monochromatic light treatment data.

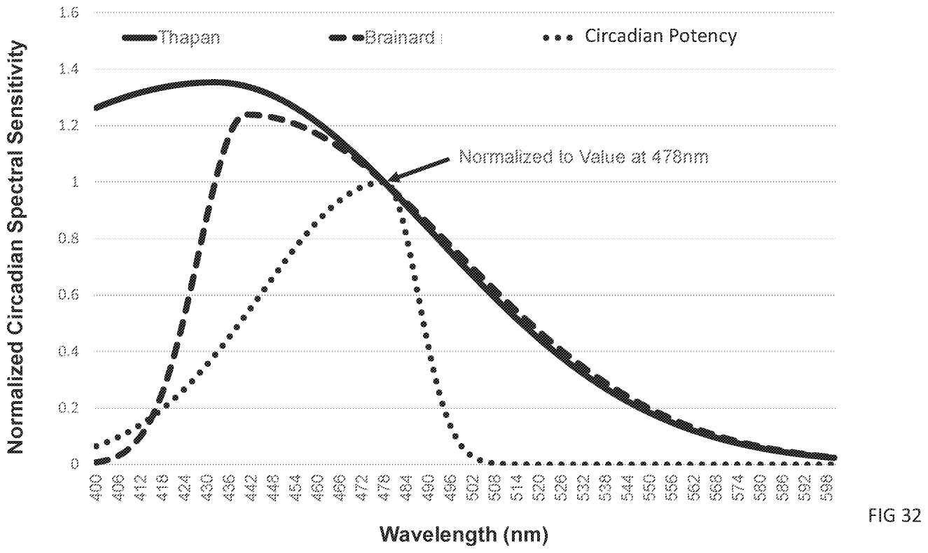

Alternatively if one assumes that the transients are additive to the underlying sustained Circadian Potency SSD determined in the initial analysis and the response to 478 nm light may be a constant then the Relative Circadian SSD would change over time of exposure as shown in FIG. 32. This result would explain the relatively faster circadian phase shifting response that is observed during the initial minutes of light exposure (Duffy and Czeisler 2009).

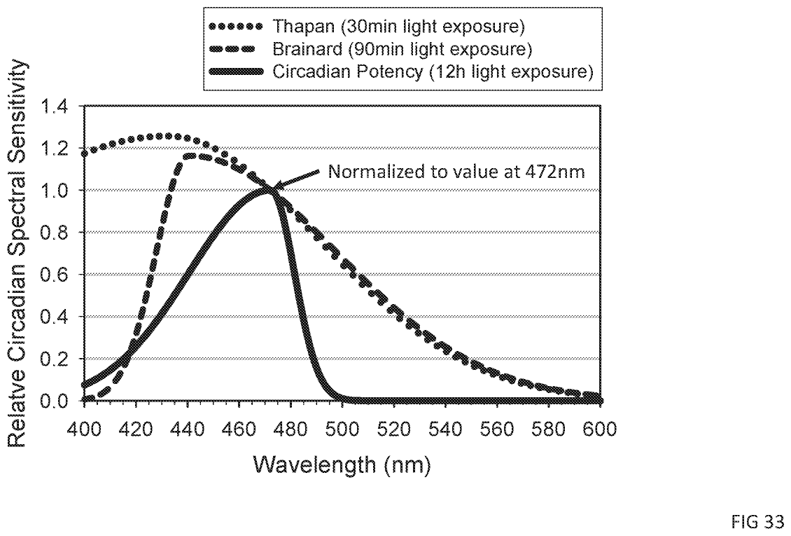

Alternatively, if one assumes that the transients are additive to the underlying sustained Circadian Potency SSD determined in the more comprehensive subsequent analysis and the response to 472 nm light may be a constant then the Relative Circadian SSD would change over time of exposure as shown in FIG. 33. This result would similarly explain the relatively faster circadian phase shifting response that is observed during the initial minutes of light exposure (Duffy and Czeisler 2009).

The Circadian Potency SSD derived for extended exposures to polychromatic white light at normal levels of illumination is also distinctly different from the Circadian Stimulation (CS) Relative Circadian SSDs for cool CCT as shown in FIG. 26, the Circadian Stimulation (CS) Relative Circadian SSDs for warm CCT as shown in FIG. 27 and the Equivalent Melanopic Lux (EML) Relative Circadian SSD as is shown in FIG. 28. The CS curve has peak sensitivity at 464 nm in cool (>approximately 3300K CCT) and 490 nm in warm (<approximately 3200K CCT) lights, and the EML has a peak sensitivity at 490 nm, whereas the Circadian Potency SSD has a peak sensitivity at 478 nm in the initial analysis, and at 472 nm in the more comprehensive subsequent analysis.

An important distinction between the Circadian Potency SSD function and the CS and EML functions is that the Circadian Potency SSD in the initial, and also in the more comprehensive subsequent, analysis shows less than 5% maximum response at 500 nm, whereas the CS (warm) shows 60% maximum response at 500 nm and the EML shows 95% of maximum response at 500 nm. The EML and CS curves therefore incorrectly predict significant Circadian Disruption by white light wavelengths above 500 nm, which may result in the design of suboptimal lights or inaccurate circadian light sensors. The suboptimal lights designed using EML or CS models may have reduced photopic stimulation which may adversely affect color and color rendering and visual performance.

Rahman et al (2011) and Gil-Lozano et al (2016) have reported suppression of nocturnal melatonin release in subjects illuminated by polychromatic white light for 12 hours at night at workplace levels of light intensity (600-1000 lux table top). However, they found that when cut off filters which eliminated light wavelengths above 500 nm were used there was a near complete restoration of melatonin levels, a finding that is predicted by the Circadian Potency SSDs disclosed herein. The veracity of these research findings have been challenged and not generally accepted by the academic scientific community because they cannot be predicted by the CS and EML models or by the short monochromatic light exposure data of Thapan (2001) or Brainard et al (2001). However, these results of Rahman et al (2011) and Gil-Lozano et al (2016) obtained under 12-hour exposures to workplace levels of white light are fully compatible and explained by the Circadian Potency SSD function disclosed herein which teaches that there is minimal circadian stimulus and melatonin suppression effect with light wavelengths above 500 nm.

Lighting Design and Manufacture Based on Circadian Potency SSD

The novel and previously unpredicted specific asymmetric shape of the Circadian Potency SSD is important to the optimal design and manufacture of white lighting sources including, but not limited to, LED chips, OLEDs, quantum dots, and fluorescent lights for indoor built environments such as are represented in FIG. 2, and in outdoor environments lit by electric lighting at night. Constructing any light source requires selecting complex trade-offs between the spectral elements that produce the desired color temperature, the desired perceptual appearance of whiteness, and the desired color rendering index (CRI), and the spectral elements that stimulate human alertness and performance, and those which either stimulate circadian rhythm synchronization during the day or must be removed to prevent disruption of circadian rhythms during the night.

Wherein the Relative Circadian Potency SSD optimized peak was determined to be approximately 478 nm in the initial analysis, and 472 nm in the more comprehensive subsequent analysis when the illuminated environment occupants are less than 45 years old, if the illuminated environment occupants are more than 50 years of age there is evidence that the peak Relative Circadian SSD may be shifted by up to 10 nm higher wavelengths (Najjar 2014) The same Circadian Potency SSD asymmetric function may need to be right shifted by 5-10 nm in more elderly populations of occupants.

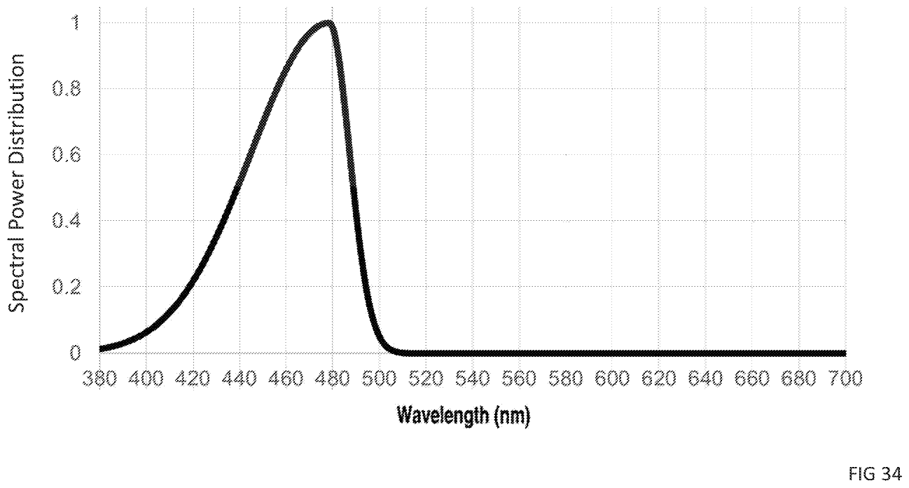

An improved light source light could be a substantially blue light that emits a circadian day optimized light (FIG. 34). The circadian day optimized light of this aspect is provided from a light source configured for emitting light having a spectral power distribution in a visible wavelength range between 380 nm and 700 nm, the spectral power distribution including a circadian day optimized peak, the spectral power distribution having a negatively skewed asymmetric distribution around the circadian day optimized peak, the circadian day optimized peak at about 472 nm or at about 478 nm, and in another embodiment, a light emitting device for emitting a circadian day optimized polychromatic white or near white light is described, the light emitting device including a light source configured for emitting light having a spectral power distribution in a visible wavelength range between 380 nm and 780 nm, the spectral power distribution including a circadian day optimized peak, and the spectral power distribution further includes greater than approximately 20% of total 380-780 nm visible irradiance in the 440-490 nm wavelength band.

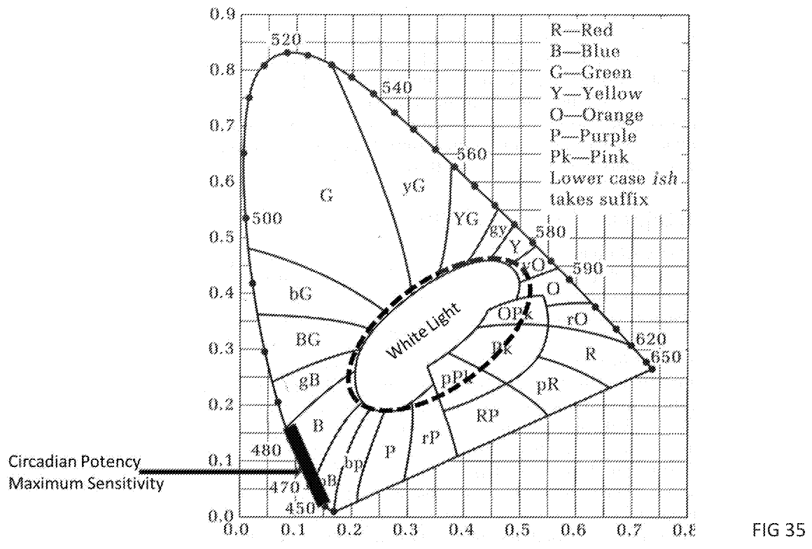

If there is no restriction on the color of the light utilized, the optimum daytime circadian simulative light is a light where the SPD of the light source matches the Circadian Potency SSD as shown in FIG. 34. This may have a peak at 472 nm or 478 nm or within the range of 470-480 nm for example. However, this light is a blue color with a location on the CIE 1931 Colorimetric chart shown by the black bar on the blue border of the CIE 1931 diagram in FIG. 35.

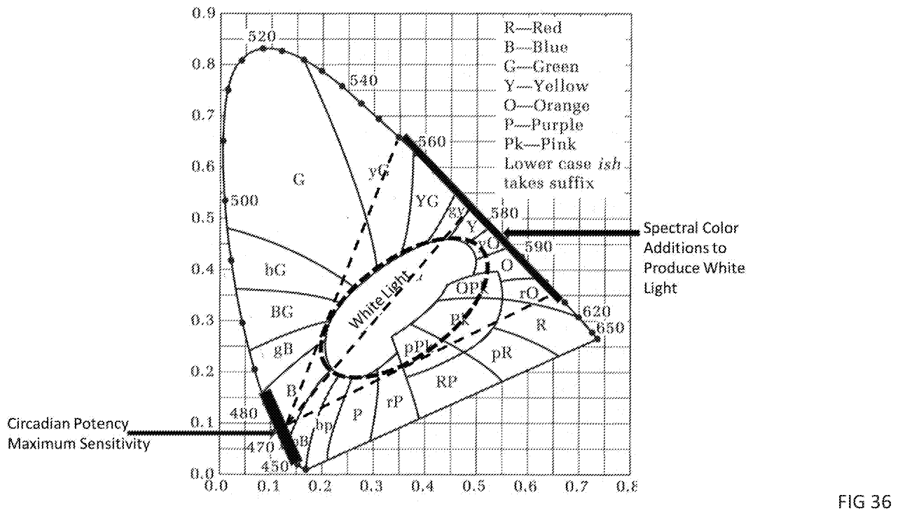

To spectrally engineer light that optimizes Circadian Potency but is white in color requires the addition of colors in the spectrum in the Green, Yellow, Orange and Red wavelengths indicated in FIG. 36 by the black bar on the right side of the CIE 1931 diagram, so that the resultant chromaticity falls with the white are marked by the white light oval.

To spectrally engineer the optimum light for night that meets the multiple objectives of 1) aesthetically pleasing white light, 2) a CCT in the 3000K to 4500K range that most users prefer, 3) a color rendering index (CRI) greater than 80, 4) human alertness and performance enhancement, and 5) prevention of Circadian Disruption by keeping Relative Circadian Potency below 6, requires access to as much of the visible light spectrum wavelengths as possible.

As 376 shows, if the requirement for example is that no wavelength in a nocturnal light SPD should provide more than 20% of maximum circadian stimulation, the Circadian Potency SSD allows over 80% of the 400-780 nm wavelengths of the visible non-UV and non IR spectrum to be available for light emissions which do not cause Circadian Disruption. In contrast the EML SSD 372 only provides 65% availability of 400-780 nm wavelengths of the visible non-UV spectrum for light emissions which do not cause Circadian Disruption and the cool CCT CS 374 only provides 53% availability of 400-780 nm wavelengths of the visible non-UV spectrum for light emissions which do not cause Circadian Disruption.

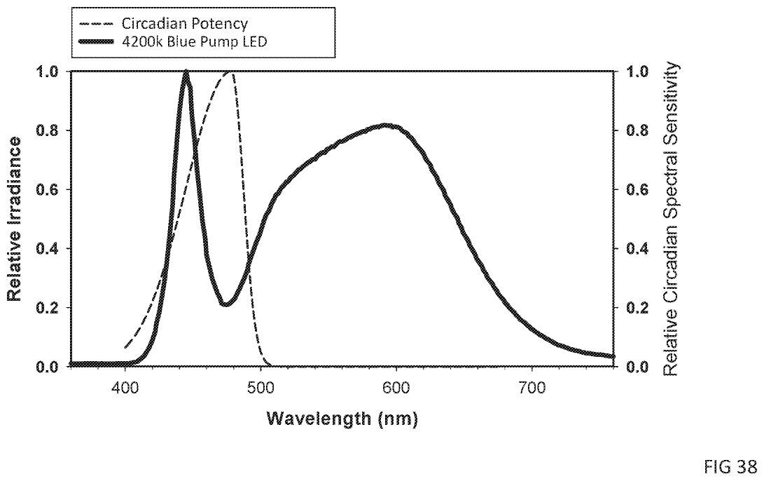

Most widely available white LEDs used in light fixtures (“Conventional White LEDs”) are designed to optimize energy efficiency (measured in lumens/watt) by utilizing a blue die with a maximum emission of about 450 nm which is coated with phosphors to create Polychromatic White Light. As can be seen in FIG. 38 this creates a SPD which is significantly sub-optimal both for day or for night circadian lighting.

In daytime applications these Conventional White LEDs have a peak emission that is at 28 nm lower wavelength than the Circadian Potency SSD peak for daytime stimulation determined in the initial analysis (FIG. 38) and 22 nm lower wavelength than the Circadian Potency SSD peak for daytime stimulation determined in the subsequent more comprehensive analysis and there is a low emission trough in the region of the 472 nm-478 nm peak sensitivity wavelengths of the circadian system according to Circadian Potency SSD Therefore, Conventional White LEDs are significantly suboptimal for use in daytime circadian synchronization and human performance.

There are several metrics that may be used to define the optimal circadian lighting for daytime use:
1) The Relative Circadian Potency,
2) the Circadian Potency in irradiance units,
3) the Circadian Potency in photon flux density units,
4) the Circadian Potency/Photopic Power Ratio (CPPPR) which expresses the Circadian Potency at any given lux level, and
5) the Percentage Blue expressed as the irradiance emitted in the range of 440-490 nm as compared to the total visible irradiance between 380-780 nm.
6) the Percentage Blue expressed as the irradiance emitted in the range of 435-485 nm as compared to the total visible irradiance between 380-780 nm

Examples of the use of these metrics to compare and select circadian optimized light sources is provided below.

The Conventional White LED provided as an example in FIG. 38 and with a CCT of approximately 4100K has a Relative Circadian Potency of approximately 21.1 which is significantly lower than the Circadian Potency of an Optimized Circadian Day White LEDs designed according to the specifications and disclosures herein which as disclosed below have a Circadian Potency of approximately 37 at similar CCT levels. High (greater than 30) Relative Circadian Potency levels boost circadian synchronization. Therefore, Conventional White LEDs are significantly suboptimal for daytime use.

This exemplar Conventional White LED in FIG. 38 with a CCT of approximately 4100K has a Circadian Potency/Photopic Power Ratio (CPPPR) of approximately 0.28 which is significantly lower than the CPPPR of an Optimized Circadian Day White LEDs designed according to the specifications and disclosures herein which as Applicants disclose herein have a CPPPR of approximately 0.50 at similar CCT levels. Therefore, Conventional White LEDs are significantly suboptimal for daytime use.

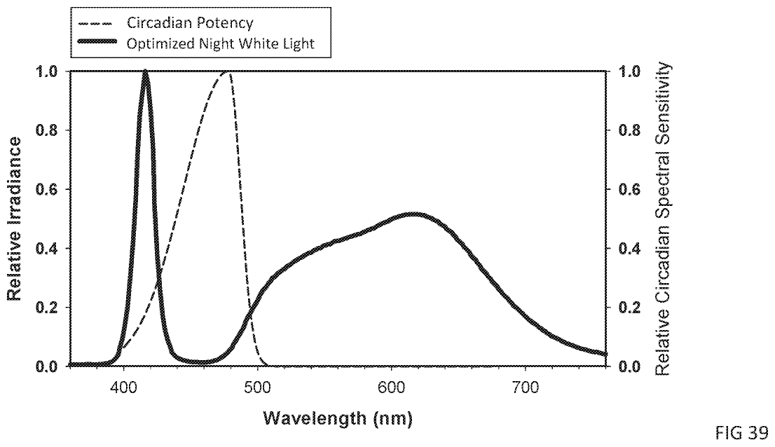

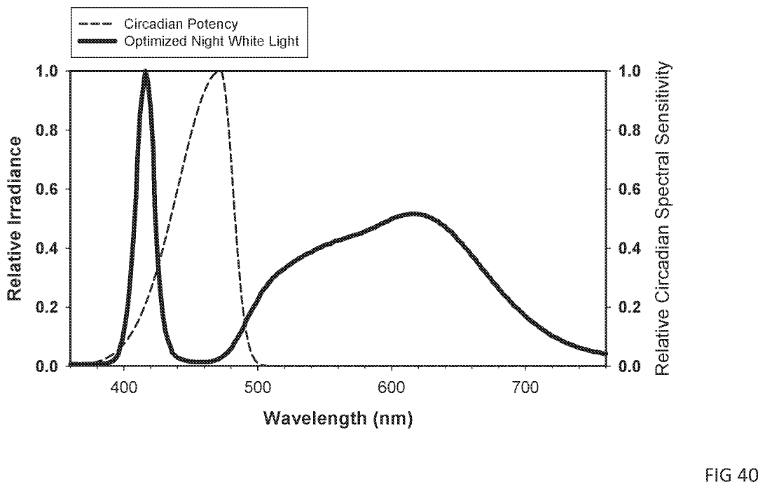

In nighttime (sunset to sunrise) applications these Conventional White LEDs emit excessive blue light at wavelength between 440-490 nm according to the Circadian Potency SSD (FIG. 38). The amount of Circadian Potent light emitted partially depends on the CCT of the light. At CCT of 6500K for example Conventional White LEDs may emit 25-30% 440-490 nm blue light, whereas at 2700K CCT they may emit 8-10% 440-490 nm blue light which are levels associated with significant circadian disruption at night. These percentage levels of blue 440-490 nm light emission are significantly greater than the 1.7% blue 440-490 nm light emission of Optimized Circadian Night White LEDs in FIG. 39 and FIG. 40 designed according to the specifications and disclosures below. Therefore, Conventional White LEDs are also significantly suboptimal for nighttime use.

The Circadian Potency of approximately 21 of the Conventional White LED illustrated in FIG. 38 is significantly greater than the Circadian Potency of an Optimized Circadian Night White LEDs illustrated in FIGS. 39 and 40 which were designed according to the specifications and disclosures herein and have a Relative Circadian Potency of approximately 5 at similar CCT levels. Therefore, Conventional White LEDs are also significantly suboptimal for nighttime use.

By precisely knowing the Circadian Potency SSD function it becomes possible for the first time to build lighting sources that maximize desirable levels of stimulation of the human circadian system during the day time hours between sunrise and sunset, and/or minimize stimulation of the human circadian system during the evening and night hours between sunset and sunrise. Using the newly disclosed Circadian Potency SSD function enables lighting engineers to maximize the circadian effectiveness while minimizing the compromises that are necessary to achieve the desired photopic, colorimetric, efficiency and other qualities of the light including CCT and the CRI.

For example, to achieve optimal white color and avoid the yellowing of light which occurs with the removal of blue wavelengths, the Circadian Potency SSD function indicates that light wavelengths less than 420 nm will have minimal influence (less than 20% of maximum stimulation) on circadian function at night. The CS function in contrast predicts 420 nm light will have 40% of maximum stimulation and thus would be unsafe and unhealthy to use at night. The Circadian Potency model disclosed here teaches the use of 400-420 nm light, and in particular, 415-420 nm light is a feature that can be included in the SPD of lights designed for nighttime (sunrise to sunset) used to provide light of an attractive white color which are depleted of blue wavelengths.

Using light wavelengths in the 415-420 nm range allows the construction of light SPDs that increase alertness and performance. (FIG. 39, 40) 420 nm light has been shown to promote and sustain significantly higher levels of alertness in human subjects than blue or red light (Revell et al 2006). This is an attractive and valuable feature of an SPD but would be predicted by the CS model to have a highly circadian disruptive effect. In contrast the Circadian Potency model disclosed here teaches this is a feature that can be included in the SPD of lights designed for nighttime (sunrise to sunset) use to increase and optimize human alertness and performance.

Using light wavelengths in the 405-420 nm range allows the construction of SPDs that kill bacterial and sterilize illuminated surfaces to prevent or minimize bacterial contamination. The CS model predicts that these SPDs would cause Circadian Disruption at night, whereas the Circadian Potency model teaches that these SPDs can be safely used at night because they will not cause significant Circadian Disruption.

As a further example, the newly disclosed Circadian Potency SSD function teaches that wavelengths of light above 500 nm may be safely used at night to improve the photopic brightness of light without disrupting circadian rhythms since the Circadian Potency SSD curve shows less than 5% maximum circadian response at 500 nm. In contrast the CS model shows 60% maximum response at 500 nm and the EML model shows 95% of maximum response at 500 nm which would indicate that light wavelengths of 500 nm could not be safely used because they would cause circadian disruption at night. Therefore, the Circadian Potency SSD function disclosed herein enables the development of light spectral power distributions with better photopic quality without compromising circadian function at night.

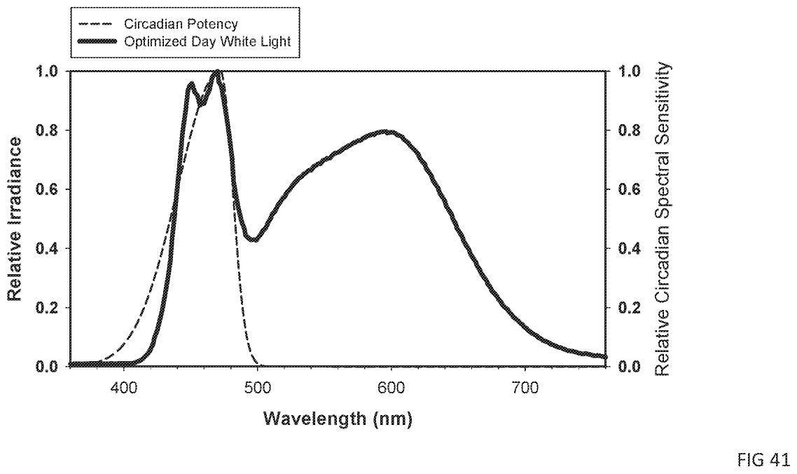

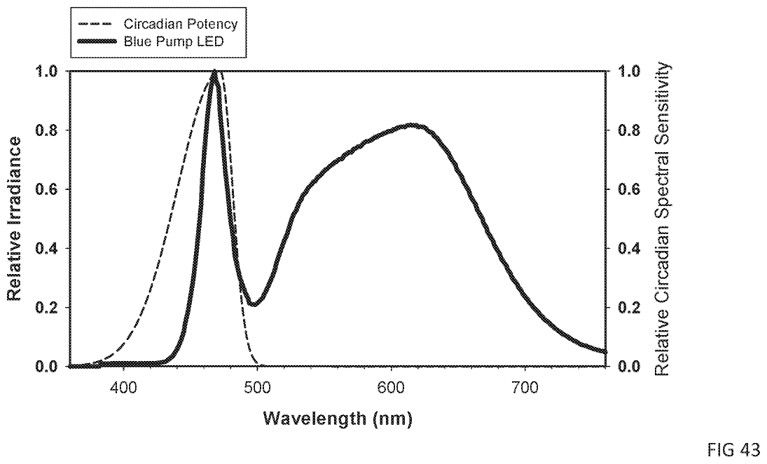

FIG. 41 and FIG. 42 and FIG. 43 illustrate examples of Optimized Circadian Day White Light source SPD which is designed using the newly disclosed Circadian Potency SSD function to maximize daytime circadian stimulation and synchronization of circadian clocks. The light source delivers a strong circadian stimulus with a peak emission at approximately 472-478 nm and provides more than 24% of the total visible ³⁸⁰⁻⁷⁸⁰ irradiance in the 440-490 nm FWHM range. Other aspects of the SPD provide the photopic stimulus to aid in the delivery of white light with good color rendering with a CRI of above 80.

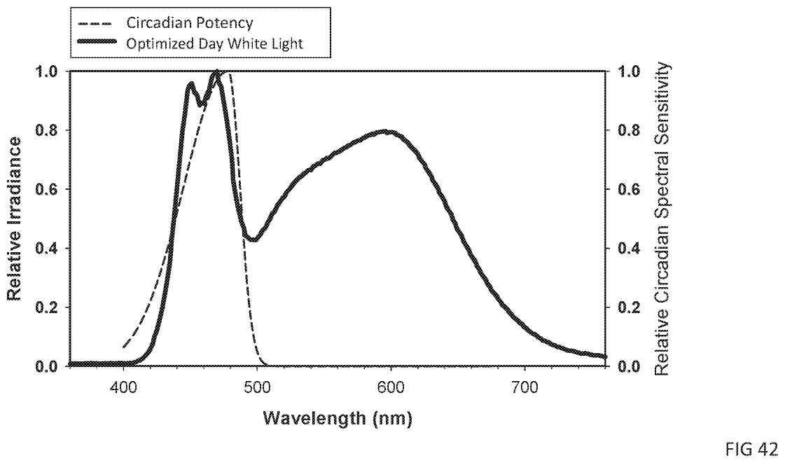

As shown in FIG. 42, the circadian potency function is established, in an embodiment, to provide a peak (e.g., an absolute peak or, in some cases, a relative peak) at around a circadian day optimized peak (e.g., 472 nm or 478 nm). In some embodiments, the distribution around the peak is adapted to more closely follow (if not be identical to) the circadian potency profile shown in dashed lines. A relative minima at about 500 nm is shown in this example, as light components beyond 500 nm are introduced to help “tune” the light such that the overall polychromatic light is an acceptable white or off-white. Accordingly, in some embodiments, the light components beyond 500 nm are balanced to establish this output. Balancing, for example, can include introducing components at different wavelengths and measuring the output to ensure that a white or off-white is provided.

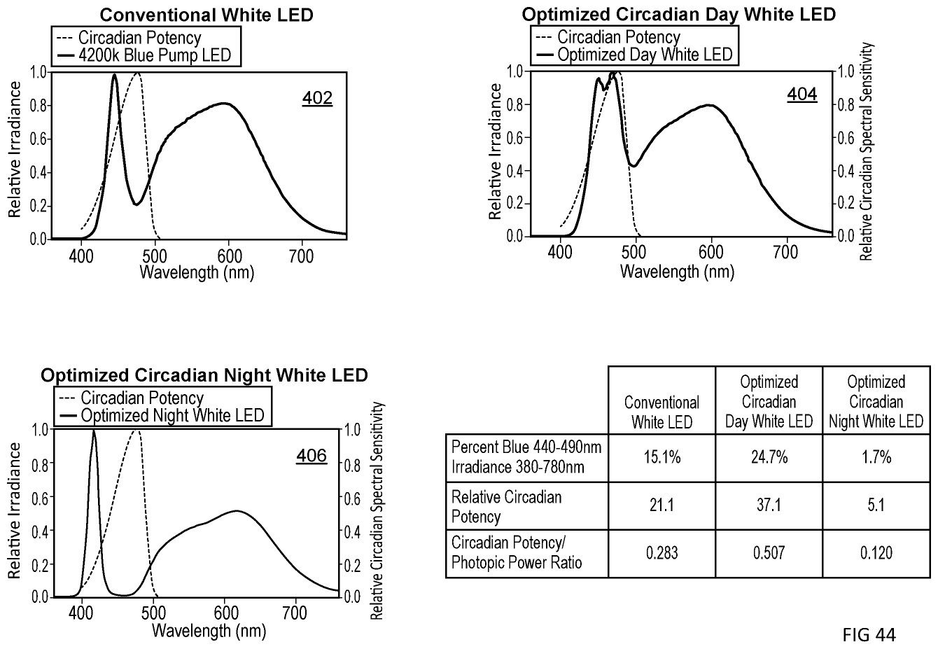

Referring to FIG. 44, this example of an Optimized Circadian Day White Light LED in 404 has a Relative Circadian Potency of 37.1 and a Circadian Potency/Photopic Power Ratio (CPPPR) of 0.507 which is significantly greater than the Relative Circadian Potency of 21.1 and CPPPR of 0.283 of Conventional White LEDs at similar CCT levels 402. A circadian night white LED distribution example is shown at 406.

In an embodiment, the spectral power distribution includes an absolute maxima at about 472 nm or at about 478 nm, and a relative minima at about 500 nm. In an embodiment, the spectral power distribution includes a relative maxima at about 600, 605, 610, or 620 nm. In an embodiment, the spectral power distribution between about 480 nm to about 700 nm balances with a power distribution around the circadian day optimized peak to provide an optimized polychromatic white light.

For the circadian night optimized polychromatic white light, a circadian night optimized trough is described. The trough can be selected from the group of wavelengths consisting of 467, 468, 469, 470, 471, 472, 473, 474, 475, 476, 477 nm, or selected from the group of wavelengths consisting of 473, 474, 475, 476, 477, 478, 479, 480, 481, 482 and 483 nm, or selected from the group of wavelengths consisting of 480, 481, 482, 483, 484, 485, 486, 487, 488, and 489 nm when illuminated environment occupants include at least one occupant whose age is greater than 50 years of age.

The circadian night optimized trough can be established such that there is a trough in a particular region, and the trough can have significant attenuation in wavelengths that are covered under the Circadian Potency SSD. In some embodiments, the trough tracks similar to an inverse of the Circadian Potency SSD. In some embodiments, the trough attenuates across the wavelengths of all or a portion (e.g., 50%, 75%, 100%) of the Circadian Potency SSD. In some embodiments, the trough has minima at 472 nm or 478 nm.

The circadian day optimized peak can include a peak at approximately 472 nm or at approximately 478 nm, or other values in the proximity, according to various embodiments. In further embodiments, characteristics of a distribution are described around the circadian day optimized peak.

The circadian night optimized trough can, in some embodiments, be similar or substantially identical to an inverse variation of the circadian day optimized peak and/or the distribution thereof.

This Circadian Potency Optimized Day White Light SPD can be generated by using a wide variety of methods which include (and are not limited to) the embodiments described below:

Adding a monochromatic blue source to an energy-efficiency (lumens per watt) optimized polychromatic white source can produce an energy-efficient optimal blue spectrum with a high Circadian Potency for daytime operation at a minimal cost. The blue source will increase the blue irradiance without significantly affecting overall illumination levels, or significantly reducing energy efficiency.

Such a light engine can be built as an LED module with two types of LEDs, a Conventional White LEDs comprising an approximately 450 nm blue pump die coated with phosphors and a monochromatic blue LED with a peak emission in the 475-480 range which has the effect of filling the Conventional White LEDs trough in emissions in the 478 nm wavelength region to create a Circadian Potency optimized SPD.

For example, as one of many possible embodiments, this SPD has been created using Cree White LEDs XPGDWT-H1-0000-00JF5 (or equivalent) with chromaticity bins: F5 Kit (4C, 4D, 5A1, 5A2, 5A3, 5A4, 5B1, 5B2, 5B3, 5B4) and selecting bins 5 and 6 of the Osram Royal Blue monochromatic LEDs that have peak emission in the 472-480 range.

Adding a monochromatic blue source to a polychromatic white source can produce optimal blue spectrum for day operation even when the user chooses to dim the light. The blue source can be used to increase the blue irradiance under dimmed overall illumination levels and thus preserve the Circadian Potency of a light source and an illuminated environment even when the user chooses to dim the lights during the daytime.

Light sources can be configured to provide a constant absolute Circadian Potency which disproportionality adjusts the power to the blue versus white LEDs to maintain the blue wavelength stimulation, or a Constant Relative Circadian Potency where the Relative SPD is maintained by proportional adjusting the power to the white LEDs and the monochromatic blue LEDs or an intermediate solution which minimizes the loss of Circadian Potency as the light is dimmed by the user.

The monochromatic blue sources and polychromatic white sources can be configured for constant blue irradiance, proportional blue irradiance or a combination of the two. Control can be open loop as well as closed loop. The controls can also integrate external inputs from sensors, control systems (e.g. building lighting management) or user controls.

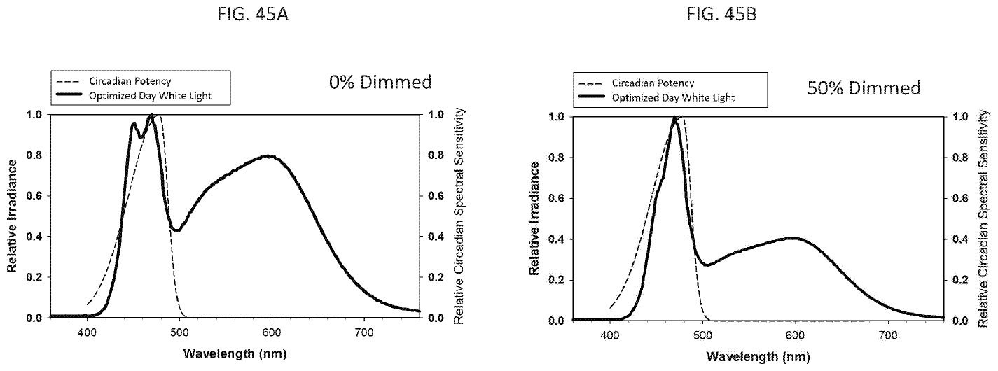

FIG. 45A and FIG. 45B illustrates an example of a light source SPD which is designed using the newly disclosed Circadian Potency SSD function with a peak at 478 nm to maximize daytime circadian stimulation and synchronization of circadian clocks even at dimmed lower light intensities using a configuration that provides constant blue irradiance. In FIG. 45A the light source is at 100% power. In FIG. 45B the light source is dimmed to 50% power.

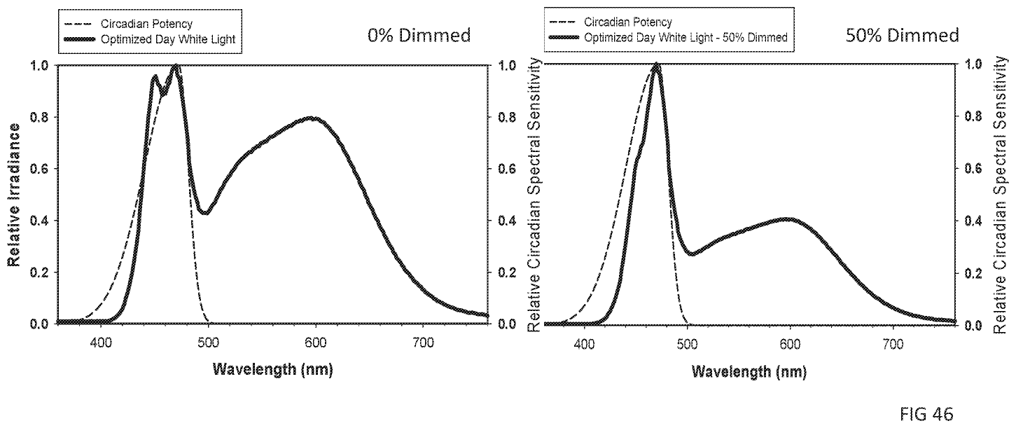

FIG. 46 illustrates an example of a light source SPD which is designed using the newly disclosed Circadian Potency SSD function with a peak at 472 nm to maximize daytime circadian stimulation and synchronization of circadian clocks even at dimmed lower light intensities using a configuration that provides constant blue irradiance. In FIG. 46A the light source is at 100% power. In FIG. 46B the light source is dimmed to 50% power.

An embodiment is a light engine comprising white LEDs and monochromatic blue LEDs which transitions from the emission spectrum in FIG. 45A or 46A to the emission spectrum in FIG. 45B or 46B when lights are dimmed. This can be achieved by reducing the power applied to the white LEDs while keeping unchanged the power applied to the 475-480 nm monochromatic blue LEDs.

At 100% power the light source has a peak emission at approximately 478 nm and provides a strong stimulus in the 440-490 nm FWHM range. Other aspects of the SPD provide the photopic stimulus to deliver white light with good color rendering with a CRI of above 80. The circadian/photopic power ratio (CPPPR) of this light source is 0.507 and the Relative Circadian Potency is 37.1.

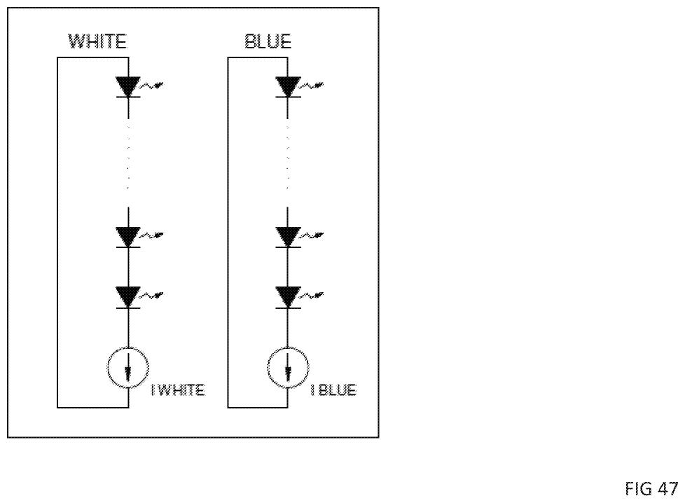

FIG. 47 depicts circuitry that maintains constant blue irradiance. One current source I_{BLUE}) maintains a constant current through the blue LEDs while the current through the white LEDs is regulated with a second current source I_{WHITE}).

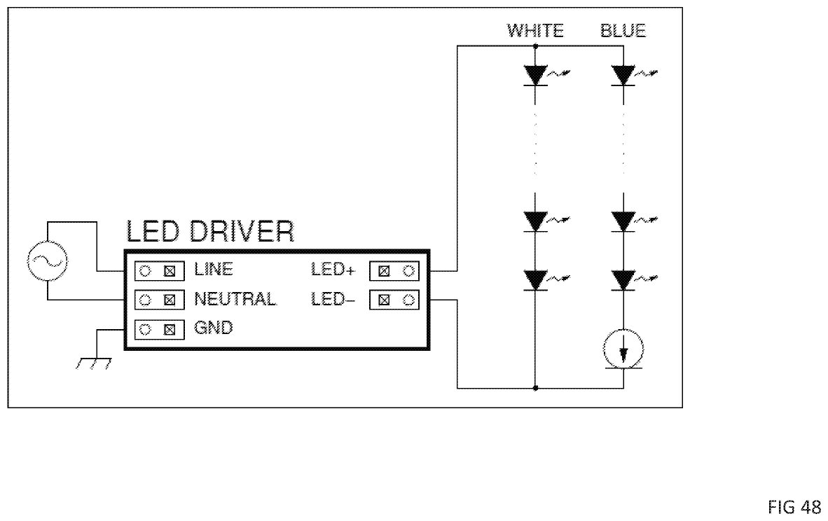

FIG. 48 depicts circuitry to maintain constant blue irradiance using a current source and a shunt current regulator. The system current is regulated by the LED driver. The current through the blue LEDs is regulated by the shunt current regulator. The current through the white LEDs is the difference of the system current and the blue LED current which is I_{WHITE}=I_{SYSTEM}-I_{BLUE}. The limitation of this design is that as I_{SYSTEM} is reduced I_{WHITE} decreases (since I_{BLUE} is constant) and the percentage of blue light increases which shifts the color temperature. As I_{WHITE}→0 the light output becomes monochromatic blue.

Another embodiment would use a current-sink instead of a current-regulator diode.

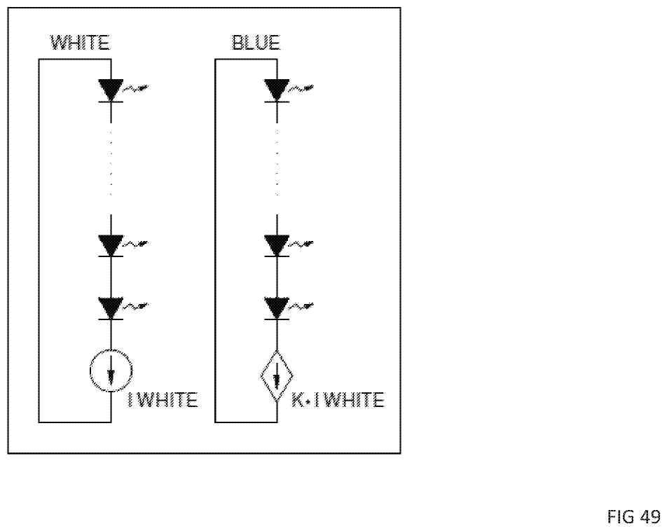

FIG. 49 depicts circuitry that maintains blue irradiance proportional to the white light output. The simplest design would set the current through the blue LED's to a value that is a fixed percentage of the current through the white LED's (i.e. I_{BLUE}=K·I_{WHITE} where K is constant.)

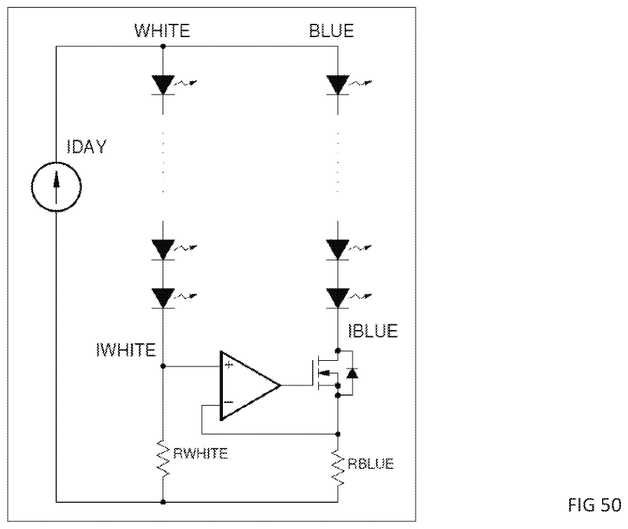

Another embodiment would control the current through the blue LEDs by sensing the current through the white LEDs and setting the blue LED current to a proportional value. FIG. 50 depicts an embodiment that uses a current sink and current shunt to perform the current regulation. In this circuit

$${I}_{BLUE} = {I}_{WHITE} \cdot \frac{{R}_{WHITE}}{{R}_{BLUE}} ( i . e . K = \frac{{R}_{WHITE}}{R\_BLUE} )$$

Another embodiment would control the current through the blue LEDs using a control signal generated by the white LED current source.

Another embodiment would control the current through the blue LEDs based on sensor data that measures the white LED irradiance.

Another embodiment would use a microcontroller to control the current through the white LEDs, calculate the value for the blue LED current and control the current through the blue LEDs.

More advanced control options are possible if K is changed from a constant to a transfer function or algorithm. For example, in a dimmable luminaire a constant blue irradiance could be maintained until the luminaire output is reduced to a minimum value. Below the minimum lumen output the control system would switch to a proportional control to maintain a maximum CCT (see Equation 1).

$${I}_{blue} = \{ \begin{matrix}{I}_{bluemax} & if {I}_{white} > {I}_{whitemin} \\ K \cdot {I}_{white} & otherwise\end{matrix}$$

Another embodiment would increase blue irradiance during the mid-morning and mid-afternoon time periods to increase alertness.

Another embodiment would change blue irradiance based on current physiological data, which may include one of more of the following: prior light exposure data, actigraphy rest activity or sleep-wake data, work-rest schedule or time and attendance data, body temperature, heart rate variability, and/or melatonin and cortisol levels (“Physiological Data”)

Another embodiment would change blue irradiance based on a schedule generated from historical physiological data.

Another embodiment would change blue irradiance to achieve a specific physiological objective (e.g. maximize performance in a physical or cognitive activity).

The luminaire could switch modes based on sensor data, external controls or user input. A closed-loop system based on a sensor that measures blue irradiance could be implemented. As blue irradiance is added from external sources (e.g. sunlight) the blue irradiance produced by the luminaire could be reduced (i.e. blue-light harvesting).

Another embodiment could change blue irradiance based on data from a sensor that measures ambient blue irradiance.

Another embodiment could change blue irradiance based on data from a wearable sensor.

Another embodiment could change blue irradiance based on user input using an external control (e.g., dimmer control).

Another embodiment could change blue irradiance based on user input using a computing device (e.g., PC or phone).

Another embodiment could change blue irradiance based on commands from a building information management system.

Another embodiment could change blue irradiance based on commands received from other luminaires.

There are other embodiments which enable the creation of the Optimized Daytime Circadian Potency SPD illustrated in FIGS. 45A and 45B.

This Optimized Daytime Circadian Potency SPD can alternatively be provided by a light engine with one type of LED comprising a die with a peak emission at 478 nm which is coated with phosphors to provide a strong photopic emission between 500 nm and 700 nm.

Another non-limiting embodiment is combining one or more green and red phosphors but not blue phosphors with high power, medium power, or low power ceramic or plastic 405 nm to 420 nm LED dies to emit white light on the 1931 CIE Chromaticity diagram with a CPPPR of 0.12 or less, a Relative Circadian Potency of 6 or less, a CCT of 2700K or greater, a CRI of 80 or greater, and blue (440 nm-490 nm) spectral content of 2% or less of Total Visible Irradiance³⁸⁰⁻⁷⁸⁰.

A non-limiting embodiment is using high power ceramic 413 nm to 420 nm LED dies with a combination of phosphors consisting of 90% 550 nm yellow phosphors and 10% 650 red phosphors that emit white light on the 1931 CIE Chromaticity diagram with a CPPPR of 0.12 or less, a Relative Circadian Potency of 6 or less, a CCT of 2700K or greater, a CRI of 80 or greater, and blue (440 nm-490 nm) spectral content of 2% or less of Total Visible Irradiance³⁸⁰⁻⁷⁸⁰.

Another non-limiting embodiment is using 413 nm to 420 LED dies with a combination of phosphors consisting of 93%-94% 540 nm green phosphors and 6%-7% 640 nm red phosphors that emit light white light on the 1931 CIE Chromaticity diagram with a CPPPR of 0.12 or less, a Relative Circadian Potency of 6 or less, a CCT of 2700K or greater, a CRI of 80 or greater, and blue (440 nm-490 nm) spectral content of 2% or less of Total Visible Irradiance³⁸⁰⁻⁷⁸⁰.

Additional non-limiting embodiments combining red and green phosphors with additional color phosphors such as phosphate or silicate based yellow phosphors, but not blue, to refine the spectrum of the emitted light while maintaining a CPPPR of 0.12 or less, and a Relative Circadian Potency of 6 or less.

This Optimized Daytime Circadian Potency SPD can be alternatively be provided by an array of Red, Green and Blue LED chips where the Blue LED has a peak emission at approximately 475-480 nm which in combination are powered to create a white light.

This Optimized Daytime Circadian Potency SPD can be alternatively be provided by an array of Red, Green and Blue and white LED chips where the Blue LED has a peak emission at approximately 475-480 nm, and which in combination are powered to create a white light.

This Optimized Daytime Circadian Potency SPD can alternatively be provided by a light engine with independently controlled Red, Green, Blue and Violet (RGBV) LEDS where only the Red, Green, and Violet LEDs are operated in night mode to create the Circadian Potency minimized SPD shown in FIG. 39 and FIG. 40.

This Optimized Daytime Circadian Potency SPD can alternatively be provided by a light engine with independently controlled Red, Green, Blue, Violet and White (RGBVW) LEDS where all LEDs can be operated in daytime mode but where only the Red, Green, and Violet LEDs are operated in night mode to create the Circadian Potency minimized SPD shown in FIG. 39.

FIG. 39 and FIG. 40 illustrate an example of a light source SPD which is designed using the newly disclosed Circadian Potency SSD function to minimize nocturnal Circadian Disruption and associated adverse effects on health (“Optimized Nighttime Circadian Potency SPD”). This optimized night (sunset to sunrise) light source has a minimum emission at approximately 478 nm and provides less than 2% of the total visible irradiance in the 440-490 nm FWHM range. Other aspects of the SPD are that is has a violet spike in emission peaking at 415-420 nm which is used to enhance the whiteness of the blue-depleted light, and also enhance its alertness boosting effectiveness. This SPD also emits light in 500-780 nm wavelengths to provide the desired photopic stimulus to enable to delivery of white light with good color rendering with a CRI of above 80.

This Optimized Nighttime Circadian Potency SPD can be generated by using a wide variety of methods which include (and are not limited to) the following embodiments:

A light engine with an Optimized Nighttime Circadian Potency SPD can be created by a White LEDs comprising a 415-420 nm violet pump die coated with red and green emitting phosphors but no blue emitting phosphors which produces little or no light emission in the 440-490 nm wavelength region and has a low Relative Circadian Potency of approximately 5 and a low Circadian Potency/Photopic Power Ratio (CPPPR) of approximately 0.12.

One embodiment which Applicants have built of the Optimized Nighttime Circadian Potency SPD uses violet dies with peak emissions in the 415-420 nm nm range that are coated by specific phosphor combinations matched to three bins (415-416, 417-418, 419-420 nm) within the peak emission range. This embodiment utilized two phosphors a 540 nm GAL (Green) and Mitsubishi 640 nm (Red). The Green phosphor was a 540 nm GAL (Aluminate) series of green phosphors supplied by Intematix. The chromaticity index color point of GAL 540 is ClEx 0.371 and CIEy 0.565. Particle size D50 (V) 11, 14.5, and 18. It has a density of 6.0 g/cubic cm and an excitation range of 200 nm to 480 nm. The red phosphor is the Mitsubishi 640 phosphor. The phosphors were used in three ratios: Green 94%: Red 6%, Green 93.5%:Red 6.5% and Red 93%:Green 7% depending on the die bin. The phosphor mix was combined with a high power 3535 ceramic die package using InGanN on silica growth architecture which emitted violet light in the 415-420 range.

LEDs with Optimized Nighttime Circadian Potency SPDs can be built including LEDs based on GaN on GaN, GaN on sapphire or GaN on silicon dies which are coated with combinations of red and green phosphors and/or other non-blue emitting phosphors.

This Optimized Nighttime Circadian Potency SPD can alternatively be provided by an array of Red, Green and Violet LED chips where the Violet LED has a peak emission at approximately 415-420 nm and which in combination are powered to create a white light with little or no emission in the 440-490 nm wavelength region.

This Optimized Nighttime Circadian Potency SPD can alternatively be provided by an array of Red, Green and Violet and white LED chips where the Violet LED has a peak emission at approximately 415-420 nm and where the white LED is covered by a filter which removes all light in the 440-490 nm wavelength band which in combination are powered to create a white light with little or no emission in the 440-490 nm wavelength region and which has little or no Circadian Potency SSD stimulus to create a night optimized SPD.

In addition to enabling the development of light sources with Optimized Daytime Circadian Potency SPDs and/or Optimized Nighttime Circadian Potency SPDs the disclosures herein on the optimized Circadian Potency SSD enable the definition of optimized control systems for circadian lighting.

Control systems are disclosed wherein the SPD of light sources may be transitioned from maximal circadian stimulation based on the newly disclosed Circadian Potency SSD function to minimal Circadian Disruption the newly disclosed Circadian Potency SSD function, based on various detected triggers.

Example triggers include at least one of the time of day, season of the year, geographical location, presence in illuminated environment, and/or chronobiological characteristics of an individual. The presence, location and/or characteristics of individuals exposed to the light sources may be determined through the use of wearable devices, facial recognition, security system logs, and expected schedules. For example, the Circadian Potency SPDs can be modified or tuned based on detected data, such as the above, or the age of the individuals. An example tuning may be to add a biasing factor, or various linear and/or non-linear modifications to the Circadian Potency SPD.

Control systems are also disclosed which take advantage of the shift in the Circadian SSD function with duration of light exposure illustrated in FIG. 30, 31, 32 or 33. Light engines may be designed with violet and blue emitting LED chips which are controlled with a timing sequence so the peak spectral emission and the SPD transitions from the 30 minute SSD function to the 90 minute SSD function to the Circadian Potency curve within two to three hours after first light exposure from a fully dark adapted state. Other embodiments are contemplated in relation to the use of a circadian optimized light in conjunction with a circadian night (CNight) optimized light.

The light emitting devices of some embodiments can be used for improving circadian potency of the light as applied to human circadian functioning of individuals exposed to the light, or for causing circadian stimulation of individuals exposed to the light, including for circadian entrainment.

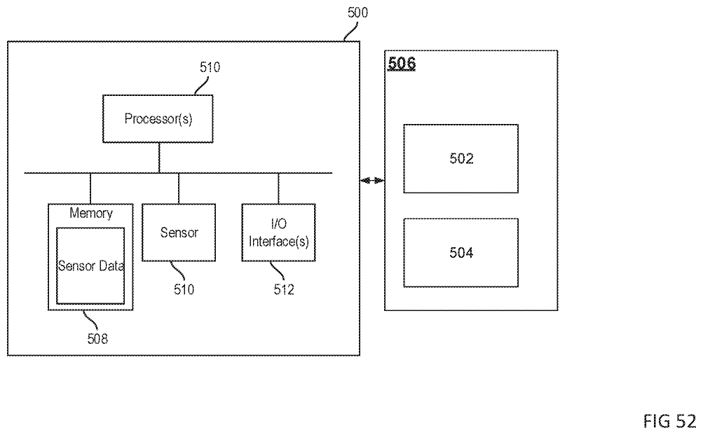

FIG. 52 is an example block diagram of an example lighting controller circuit 500, according to some embodiments.

A controller circuit 500 is described in some embodiments that is a control system that is adapted for providing either circadian day optimized polychromatic white light or circadian night optimized polychromatic white light including: a first light emitting device 502 for emitting the circadian day optimized polychromatic white light, and a second light emitting device 504 for emitting the circadian night optimized polychromatic white light, according to various embodiments. Alternatively, a lighting system is proposed for providing either circadian day optimized polychromatic white light or circadian night optimized polychromatic white light including: a light emitting device 506 configured for at least a first mode of operation and a second mode of operation, the light emitting device in the first mode configured for emitting the circadian day optimized polychromatic white light, the light emitting device in the second mode configured for emitting the circadian night optimized polychromatic white light, according to various embodiments.

The controller circuit can be configured to provide electronic signals controlling the light emitting device to toggle operation between the first mode of operation and the second mode of operation. These electronic signals can include formulated data packets including machine-interpretable instructions that can be executed by a processor, or analog electronic control values or control commands (e.g., +/−5 V signals, solenoid/switch/transistor control signals which control switching mechanisms that impede or allow the flow of electricity).

The controller circuit can be configured to toggle operation between the first mode of operation and the second mode of operation in accordance with a circadian schedule adapted for one or more users exposed to the light from the lighting system, or to entrain the circadian schedule of the one or more users. The schedules can be stored in non-transitory computer readable media or data storage 508 on the controller circuit (e.g., provided by a work-scheduler device in the form of circadian data storing data structures or data packets). For example, Zigbee™ signals can be sent to wireless controllable lightbulbs. Other protocols are possible beyond IEE 802.15.4-based specifications, and this is shown only as a non-limiting example. The electronic signals can be generated by processor 510, and the data packets can be received from interfaces 512.

The schedules can include 24 hour work periods (e.g., night shift/day shift/evening shift), instructions per hour, targeted schedules (e.g., for airplanes or vehicles where there is transition between time zones), to account for changes in time zones due to daylight savings times, among others. The schedules are used to transition between the day mode of operation and the night mode of operation, which a single lighting source can be toggled to modify operation or different lighting sources can be turned on/off or dimmed for transition. For example, the lighting system can be provided on a vehicle travelling from an origin region to a destination region, and the circadian schedule is adapted to entrain the circadian functioning of the one or more users exposed to the light from the lighting system based on a time zone of one of the origin region and the destination region. The controller circuit can include a printed circuit board, a field programmable gate array, among others. The data storage 508 can include solid state memory, random access memory, read only memory, among others.

The controller circuit 500 can be electronically coupled to the lighting devices. In some embodiments, the controller circuit is also coupled to a work scheduler server, such as a computer housing an enterprise resource planning (ERP) software that tracks work shifts (e.g., tracking airline crew schedules).

Other embodiments are contemplated in relation to the use of a circadian day optimized light (CDay) in conjunction with a circadian night (CNight) optimized light, which may be selectively activated depending on a type of desired circadian effect (e.g., stimulus or protection).

Dimmer variations are also contemplated whereby the overall amount of power in the light is adjusted or controlled in relation to a dimming effect being generated by the control of the light. For example, during a time when circadian stimulation is desired, although the CDay light is being dimmed, to maintain effective power at the circadian optimized peak, the circadian potency is managed and the SPD of the light may be adjusted to account for the increased kurtosis of the circadian optimized peak (e.g., to deliver a consistent amount of irradiance despite the dimming).

Dimmer variations are also contemplated whereby the overall amount of power in the light is adjusted or controlled in relation to a dimming effect being generated by the control of the light during a time when circadian stimulation is desirable.

For example, although the light is being dimmed, to maintain effective power at the circadian day optimized light (CDay) peak, the circadian potency is managed (e.g., tracked, controlled, maintained) and the SPD of the light may be adjusted to account for the increased kurtosis of the circadian optimized peak (e.g., to deliver a consistent amount of irradiance despite the dimming). The CDay light may include compensation mechanisms to maintain the overall color of the light as substantially white.

A circadian night optimized light can be provided that is specially configured for attenuating frequencies of light in accordance with an inverse of the Circadian SSD function (e.g., attenuating light especially around the circadian-optimized peak of the SSD function, in a negatively skewed distribution).

The circadian night optimized light can include one or more violet light emitting devices that increase an intensity of light at violet wavelength ranges of approximately 410 nm to approximately 430 nm to compensate for a reduction or attenuation of light in accordance with the Circadian Potency SSD (e.g., a reduction in light at night where light under the Circadian Potency SSD SPD curve is removed or otherwise attenuated). This reduction may be conducted using filters or tunable lighting components.

Without compensation, the light may appear unduly orange or yellow. Accordingly, a substantially white light for the night circadian functioning optimized polychromatic light can be provided despite the reduction or attenuation of light in accordance with the circadian-day optimized spectral power distribution.

The light sources may be controlled and/or operated through various means and/or systems that may range from fully automated digital systems to analog systems controlling the light sources through the control of power provided to the light sources. In some embodiments, the lights sources may be controlled through the use of one or more control systems, which may send instructions and/or other command signals to the light sources. The one or more control systems may be implemented using, for example, computing devices having non-transitory computer readable media and/or various data interfaces. The one or more control systems may include servers, and may be implemented on various technologies and platforms.

In addition to enabling the development of light sources and control systems with Optimized Daytime Circadian Potency SPDs and/or Optimized Nighttime Circadian Potency SPDs the disclosures herein on the optimized Circadian Potency SSD enable the definition of optimized light sensing systems for circadian lighting.

Controller circuits 500 can be utilized to control the transition of lighting from the circadian night optimized light and the circadian day optimized light. In some embodiments, a single combined lighting is provided that is tunable between spectral profiles associated with the circadian night optimized light and the circadian day optimized light. Tune-ability can include the use of filters, tunable phosphor designs, light emitting devices having different lighting profiles responsive to control outputs (e.g. analog or digital signals)

In other embodiments, a lighting system is provided that has separate circadian night optimized lights and the circadian day optimized lights, and a controller circuit 500 is utilized to transition between the use of the separate circadian night optimized lights and the circadian day optimized lights. Transition can be on/off for each, or may be a controlled mix of power provided to each (e.g., gradual dimming/powering up). The control may be established based on circadian states being tracked (either desired circadian states for entrainment or maintenance of existing circadian states).

Circadian light sensing apparatus and/or systems are disclosed based on the newly disclosed Circadian Potency SSD function which accurately assess the circadian stimulus in any polychromatic white light source at the light intensities used in workplaces and homes and other indoor and outdoor environments. The use of these sensing apparatus and/or systems 510 enables the design and construction of improved light sensing systems to ensure lights in a built environment are achieving desired circadian health and performance outcomes for the occupants.

Sensing systems 510 can include light sensors, such as networked lux meters, etc., which track not only the power of light, but also the spectral makeup of light. The circadian stimulating properties can be tracked in accordance with various control systems, and the overall circadian effect based on the Circadian Potency SSD can be tracked for various individuals exposed to the light. Accordingly, the Circadian Potency SSD and controllable lighting thereof can be utilized in conjunction with access management or timesheet systems to more accurately track and manage circadian functioning of individuals within, for example, a workstation. Circadian Potency SSD can be optimized based on desired circadian timings, and error values and deviations can be tracked.

In another embodiment, the spectral power distribution for circadian functioning is tracked by a configured light measuring device, which tracks a quantity of incident light having a spectral power distribution in a visible wavelength range between 380 nm and 700 nm to detect the quantity of incident light that is provided within the Circadian Potency SSD for circadian functioning.

Accordingly, a circadian-specific light meter 510 is provided. The light meter 510 is specifically designed for tracking the Circadian Potency SSD function. Example hardware technology that can be utilized for tracking the Circadian Potency SSD function include an array of neural density filters having specific densities that receive incident light and measure the amount of photopower provided from each of the filters. A photosensor can be coupled to the filters to measure the quantity and/or power of light. In another embodiment, photovoltaic photosensors are utilized that generate voltages proportional to light exposure. For example, photosensors can include photoresistors, and can include silicon or Cadmium Sulfide sensor technologies. The light meter 510 can be an individual/workstation specific device, such as a necklace, a wrist worn device, a wall device, a desktop device, among others. The light meter can also be integrated as a retrofit into other devices, such as wall clocks, televisions, among others.

Light is not necessarily incident in all embodiments. For example, in a workplace, the light meter can include reflected light meters, among others. Where incident light is being tracked, an integrating sphere may be utilized to normalize for differences in reflectance between surfaces.

In another embodiment the spectral power distribution signature of a room, workplace or other indoor space can be expressed as a conversion factor which translates the measurement of the spectral power distribution at a fixed point in the room (e.g. horizontal illuminance at table top) into the spectral power distribution in vertical illuminance at the level of the cornea of the eye for a person oriented in a typical working position in the workplace. The spectral distribution signature example in FIG. 45A and FIG. 45B is calculated by measuring the absolute irradiance in a given lighting environment at 1 nm intervals using a spectrophotometer in two positions (a) horizontal illuminance at table top and b) vertical illuminance at the cornea of a person sitting in a chair facing the table, and then for every 1 nm bin of the SPD expressing the vertical irradiance as a fraction of the horizontal irradiance, as obtained by sensor 510.

The SPD of a polychromatic white light source in an indoor space will be modified before it reaches the eyes of a room occupant by the spectrally specific properties of surfaces which absorb, reflect and/or fluoresce light in that indoor space. The conversion factor is determined by the spectral absorption, reflectance and/or florescence of the light of the surfaces in the field of view of the human subject. For example, certain white paints have brightening components which absorb violet light and fluoresce blue light. Other wall colors like yellow absorb blue and other spectral wavelengths and reflect yellow. Each modify the SPD of light reaching the eyes of the room occupants.

In some embodiments the light measurement device in some embodiments may incorporate and utilize Circadian Potency conversion factor algorithms which enable the Spectral Power Distribution (SPD) of light sources in a room to be measured at a fixed point in the room (e.g. table top, floor, or wall or ceiling or light fixtures) and to be converted into the active effective SPD and Circadian Potency of the vertical illuminance at the room occupant's eyes. The biologically relevant spectral and intensity characteristics of light is the light entering the room occupant's eyes and the conversion factors enable the Circadian Potency of the lighting environment to be estimated without requiring room occupants to use wearable devices to record light spectral exposure.

In another embodiment, the light meter is configured to determine light levels in accordance with the Circadian Potency SSD function based on photographic or video data, which can be used, for example, in a workplace or operating environment to review tracked historical data to assess circadian functioning effects based on expected exposure in accordance with the Circadian Potency SSD function for individuals in the workspace or operating environment. The light meter 510 can be coupled with workplace fatigue monitoring systems, which may automatically modify crew scheduling or enforce rest periods based on light meter readings. This is especially important where fatigue or microsleep lead to safety errors and injury events (e.g., truck driver, forklift operator, pilot, power plant workers).

A controller circuit 500 may receive the light measurements in the form of electronic signals, and in some embodiments, the controller circuit may capture readings in wavelength designated “bins” (e.g., specific ranges), which are then associated with different weighting based on the spectral power distribution for circadian functioning as described herein. Specific bins may be established for discrete wavelength ranges, such as groups of 1, 3, 5 nm ranges, and correspond to the Circadian Potency SSD function described herein.

The combined effect on circadian functioning can be determined based on the weighted effect from the various bins. Weighting may be based, for example, on the Circadian Potency SSD function, with greater weightings provided to those bins close to the circadian peak. In some embodiments, weightings are established in accordance with the skewed spectral profile of the Circadian Potency SSD function. However, it is important to recognize that fatigue management may require the consideration of multiple factors, and accordingly, the light meter may provide an input into a holistic fatigue tracking system as fatigue is a complex phenomenon which may have contributions from non-light-based sources, such as sleep deprivation, REM vs. non-REM sleep, activity levels, nutrition, etc.

In a specific, non-limiting example, an airline may track light exposure to crew members using 510, for example, and automatically attempt to entrain circadian rhythms in an attempt to prevent fatigue effects as the crew members transition across time-zones. Sensory devices may be on-board and may track incident lighting that flows through the windows, and where there is overstimulation or under stimulation of the circadian systems of the individuals detected, the crew members may be automatically flagged for increased rest requirements. Overstimulation or under stimulation may be tracked, for example, by comparing adherence to a desired amount of stimulation through the assessment of summed error values, among others. A potential control mechanism may be a feedback loop system adapted for minimizing error over time.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized.

Accordingly, the appended claims are intended to include within their scope such processes, machines, manufacture, compositions of matter, means, methods, or steps.

As can be understood, the examples described above and illustrated are intended to be exemplary only.

### REFERENCES

Brainard G C, Hanifin J P, Greeson J M, Byrne B, Glickman G, Gerner E and Rollag M D (2001). Action spectrum for melatonin regulation in humans: evidence for a novel circadian photoreceptor. J Neurosci 21 (16): 6405-6412
Clark E, Lesniak N (2017). Circadian Lighting Solutions Are Real and Important—Why Aren't They Being Used? Metropolis. https://www.metropolismag.com/design/circadian-lighting-survey/
Figueiro M G (2017). Disruption of Circadian Rhythms by Light During Day and Night. Curr Sleep Med 3(2):76-84.
Gage et al (1977 in Schubert (2016). Light-emitting diodes. https://www.ecse.rpi.eduhschubert/Light-Emitting-Diodes-dot-org/2006%20LED %20book %20pictures-Standard %20quality %20(30%20MB).pdf
Gil-Lozano M, Hunter P M, Behan L A, Gladanac B, Casper R F, Brubaker P L (2016). Short-term sleep deprivation with nocturnal light exposure alters time-dependent glucagon-like peptide-1 and insulin secretion in male volunteers. Am J Physiol Endocrinol Metab 310(1):E41-50.
Gooley J J, Rajaratnam S M, Brainard G C, Kronauer R E, Czeisler C A and Lockley S W (2010). Spectral responses of the human circadian system depend on the irradiance and duration of exposure to light. Sci Transl Med 2(31): 31 ra33
Lucas R J, Peirson S N, Berson D M, Brown™, Cooper H M, Czeisler C A, Figueiro M G, Gamlin P D, Lockley S W, O'Hagan J B, Price LLA, Provencio I, Skene D J, and Brainard G C (2014). Measuring and using light in the melanopsin age. Trends Neurosci 37(1): 1-9
Najjar R P, Chiquet C, Teikari P, Cornut P L, Claustrat B, Denis P, Cooper H M Gronfier C (2014) Aging of Non-Visual Spectral Sensitivity to Light in Humans: Compensatory Mechanisms. PLOS: Jan. 23, 2014 https://doi.org/10.1371/journal.pone.0085837
Rahman S A, Marcu S, Shapiro C M, Brown T J, and Casper R F (2011) Spectral modulation attenuates molecular, endocrine, and neurobehavioral disruption induced by nocturnal light exposure. Am J Physiol—Endocrinology and Metabolism 300(3): E518-E527
Reiter R J, Tan D X, Galano A (2014). Melatonin: exceeding expectations. Physiology 29(5):325-33
Revell V L, Arendt J, Fogg L F and Skene D J (2006). Alerting effects of light are sensitive to very short wavelengths. Neurosci Lett 399(1-2): 96-100
Thapan K, Arendt J and Skene D J (2001). An action spectrum for melatonin suppression: evidence for a novel non-rod, non-cone photoreceptor system in humans. J. Physiol 535: 261-267

1. A light emitting device for emitting a circadian day optimized light, the light emitting device comprising:
a light source configured for emitting light having a spectral power distribution in a visible wavelength range between 380 nm and 700 nm, the spectral power distribution including a circadian day optimized peak, the spectral power distribution having a negatively skewed asymmetric distribution around the circadian day optimized peak, the circadian day optimized peak at a wavelength between about 472 nm to about 478 nm.

2. The light emitting device of claim 1, wherein the circadian day optimized peak is at about 472 nm or at about 478 nm.

3. The light emitting device of claim 1, wherein the circadian day optimized peak is at about 472 nm, and 76% of spectral power is emitted within a full width half maximum (FWHM) range of the circadian day optimized peak between about 435 nm to about 483 nm.

4. The light emitting device of claim 1, wherein the circadian day optimized peak is at about 478 nm.

5. The light emitting device of claim 1, wherein the negatively skewed asymmetric distribution is defined by the relation:
$$P ( λ ) = \frac{1}{σ * √ 2 π} * {e}^{- \frac{{( λ - μ )}^{2}}{2 {σ}^{2}}}$$
wherein σ is a standard deviation, λ is a wavelength, and μ is a mean set at the circadian day optimized peak, and separate values for σ are applied at different sides of the negatively skewed asymmetric distribution.

6. The light emitting device of claim 1, wherein the negatively skewed asymmetric distribution substantially fits along a curve defined by the relations:
$$P ( λ ) = \frac{1}{33.5 * √ 2 π} * {e}^{- \frac{{( λ - 478.3 )}^{2}}{2 * {33.5}^{2}}} ( left side of the peak )$$
$$P ( λ ) = \frac{1}{8.9 * \sqrt{2 π}} * {e}^{- \frac{{( λ - 478.3 )}^{2}}{2 * {8.9}^{2}}} ( right side of the peak ) , \\ or the relations \text{:}$$
$$P ( λ ) = \frac{1}{31.7 * √ 2 π} * {e}^{- \frac{{( λ - 472.2 )}^{2}}{2 * {31.7}^{2}}} ( left side of the peak )$$
$$P ( λ ) = \frac{1}{9.2 * \sqrt{2 π}} * {e}^{- \frac{{( λ - 472.2 )}^{2}}{2 * {9.2}^{2}}} ( right side of the peak ) .$$

7. The light emitting device of claim 5, wherein a left side of the negatively skewed asymmetric distribution has a standard deviation (σ) value of 30, 31, 32, 33, 34, or 35.

8. The light emitting device of claim 5, wherein a right side of the negatively skewed asymmetric distribution has a standard deviation (σ) value of 7, 8, 9, 10, or 11.

9. The light emitting device of claim 1, wherein the negatively skewed asymmetric distribution includes at least 78% of an area under a curve defined by the spectral power distribution in the visual wavelength range between about 440 and about 490 nm.

10. The light emitting device of claim 1, wherein the negatively skewed asymmetric distribution is a mirror image log-normal distribution, or the negatively skewed asymmetric distribution is comprised of a first half-Gaussian function and a second half-Gaussian function.

11. The light emitting device of claim 1, wherein the circadian day optimized light is substantially blue, and only includes light in a wavelength range between approximately 380 nm and approximately 505 nm.

12. The light emitting device of claim 1, wherein the spectral power distribution further includes supplemental polychromatic light provided in the 480-700 nm wavelength range, the supplemental polychromatic light adapted such that the light provided by the light emitting device is substantially white or near-white.

13. A light emitting device for emitting a circadian day optimized polychromatic white or near white light, the light emitting device comprising:
a light source configured for emitting light having a spectral power distribution in a visible wavelength range between 380 nm and 780 nm, the spectral power distribution including a circadian day optimized peak; and
wherein the spectral power distribution further includes greater than approximately 20% of total 380-780 nm visible irradiance in the 440-490 nm wavelength band.

14. The light emitting device of claim 13, wherein the polychromatic white or near white light is provided within an oval space on the CIE 1931 chromaticity diagram with the coordinates of the long axis between x=0.47, y=0.45 and x=0.21, y=0.26, and coordinates of the short axis between x=0.31, y=0.40 and x=0.37, y=0.30.

15. The light emitting device of claim 13, wherein the polychromatic white or near white light is provided defined according to the ANSI standard C78.377-2008 using the Planckian locus or black body line where the white light area for the range of CCTs between 2700K to 6500K is described by the length of the white CCT lines which are each 0.012 Duv in length, and are +−0.006 Duv from the Planckian locus.

16. The light emitting device of claim 13, wherein the circadian day optimized peak is selected from the group of wavelengths consisting of 467, 468, 469, 470, 471, 472, 473, 474, 475, 476, 477 nm.

17. The light emitting device of claim 16, wherein the circadian day optimized peak is at about 472 nm.

18. The light emitting device of claim 13, wherein the circadian day optimized peak is selected from the group of wavelengths consisting of 473, 474, 475, 476, 477, 478, 479, 480, 481, 482 and 483 nm.

19. The light emitting device of claim 18, wherein the circadian day optimized peak is at about 478 nm.

20. The light emitting device of claim 13, wherein the circadian optimized peak is selected from the group of wavelengths consisting of 480, 481, 482, 483, 484, 485, 486, 487, 488, and 489 nm when illuminated environment occupants include at least one occupant whose age is greater than 50 years of age.

21-53. (canceled)
